(19) United States
(12) Patent Application Publication
(10) Pub. No.: US 2025/0084390 A1
(43) Pub. Date: Mar. 13, 2025

(54) RECRUITMENT OF DNA POLYMERASE FOR TEMPLATED EDITING

(71) Applicant: Pairwise Plants Services, Inc., Durham, NC (US)

(72) Inventors: Jingyi Nie, Durham, NC (US); Aaron Hummel, Hillsborough, NC (US); Shai Joshua Lawit, Durham, NC (US)

(21) Appl. No.: 18/941,140

(22) Filed: Nov. 8, 2024

Related U.S. Application Data
(63) Continuation of application No. 17/142,570, filed on Jan. 6, 2021, now Pat. No. 12,173,335.
(60) Provisional application No. 62/957,542, filed on Jan. 6, 2020.

(51) Int. Cl. C12N 9/16; C12N 15/113

(52) U.S. Cl. CPC C12N 9/16; C12N 15/113; C12N 2310/20; C12N 2310/3513; C12N 2310/3517; C12N 2310/3519; C12Y 301/00

(57) ABSTRACT

This invention relates to recombinant nucleic constructs comprising a sequence-specific DNA binding protein, DNA-dependent DNA polymerase and a DNA encoded repair template, optionally a DNA endonuclease or wherein the sequence-specific DNA binding protein comprises DNA endonuclease activity, and methods of use thereof for modifying nucleic acids in cells and organisms.

### STATEMENT OF PRIORITY

This application is a continuation of U.S. application Ser. No. 17/142,570, filed on Jan. 6, 2021, which claims the benefit, under 35 U.S.C. § 119(e), of U.S. Provisional Application No. 62/957,542 filed on Jan. 6, 2020, the entire contents of each of which are incorporated by reference herein.

### STATEMENT REGARDING ELECTRONIC FILING OF A SEQUENCE LISTING

A Sequence Listing in XML format, entitled 1499-14CT_ST26.xml, 219,551 bytes in size, generated on Aug. 28, 2024, and filed herewith, is hereby incorporated by reference into the specification for its disclosures.

### FIELD OF THE INVENTION

This invention relates to recombinant nucleic constructs comprising a sequence-specific DNA binding protein, DNA-dependent DNA polymerase and a DNA encoded repair template, optionally a DNA endonuclease or wherein the sequence-specific DNA binding protein comprises DNA endonuclease activity, and methods of use thereof for modifying nucleic acids in cells and organisms.

### BACKGROUND OF THE INVENTION

Precise, templated editing typically involves introducing a double strand break (DSB) in the target site and providing a template with the desired edits to be incorporated. The incorporation of the sequence from an edit template to a target site relies on templated repair of DSB through homologous recombination pathway, which is not a dominant pathway for DNA repair in most eukaryotic cells. In addition, the endogenous homologous recombination pathway is a complex process with multiple steps, each of which have inherent bottlenecks and can be difficult to manipulate. Overall, efficiency of homologous recombination mediated templated editing is typically low in human cells, and even lower in plant cells due to the low efficiency of reagent delivery and difficulty in recovering edited plants.

The best templated editing efficiencies in eukaryotes other than yeast have been accomplished in human cell culture where the delivery of a cocktail of reagents (e.g., a DNA endonuclease or nickase, a repair template, NHEJ inhibitors, HDR stimulators) can be readily coordinated and with high efficiency. Specifically, in human cells, precise templated editing has been demonstrated using a complex of three components: 1) a nickase that can be recruited to sequence specific site by a guide RNA; 2) a guide RNA with extended sequence that binds to the 3′ of nicked DNA and encodes repair template with desired edits; and 3) a RNA dependent DNA polymerase (reverse transcriptase) fused to the nickase, which uses the 3′ end of the nicked DNA and a primer to synthesize DNA (e.g., incorporate the edit). In certain human cell types, up to 50% of precise templated editing has been reported (Anzalone et al. Nature 576:149-157(2019)).

Unlike in human cells, in plants, delivery multiple reagents in different compositions can be difficult. It can also be difficult to deliver high doses of repair template, which can improve templated editing efficiency by increasing the availability of the repair template in the cell. To date, the majority of templated editing successes in plants have been achieved by particle bombardment of DNA expression cassettes and repair templates. The best editing efficiencies are in the range of less than 10%, with many studies being less than 1%. The highest efficiencies reported are often only at specific repair loci in the genome with no or a poor understanding of a mechanism that might lead to higher efficiencies of HDR.

### SUMMARY OF THE INVENTION

One aspect of the present invention provides a first complex comprising: (a) a first sequence-specific DNA binding protein that is capable of binding to a first site on a target nucleic acid; and (b) a first DNA-dependent DNA polymerase.

A second aspect of the invention provides a first complex comprising: (a) a first sequence-specific DNA binding protein that is capable of binding to a first site on a target nucleic acid and comprises endonuclease activity that is capable of introducing a single stranded nick or a double strand break; (b) a first DNA-dependent DNA polymerase; and (c) a first DNA encoded repair template.

A third aspect of the invention provides a first complex comprising: (a) a first sequence-specific DNA binding protein that is capable of binding to a first site on a target nucleic acid; (b) a first DNA-dependent DNA polymerase; (c) a first DNA endonuclease; and (d) a first DNA encoded repair template.

A fourth aspect of the invention provides a second complex comprising: (a) a second sequence-specific DNA binding protein that is capable of binding to a second site on a target nucleic acid; and (b) a DNA-encoded repair template.

A fifth aspect of the invention provides an engineered (modified) DNA-dependent DNA polymerase fused to an affinity polypeptide that is capable of interacting with a peptide tag or an RNA recruiting motif.

A sixth aspect of the invention provides an RNA molecule comprising (a) a nucleic acid sequence that mediates interaction with a CRISPR-Cas effector protein; (b) a nucleic acid sequence that directs the CRISPR-Cas effector protein to a specific nucleic acid target site through a DNA-RNA interaction, and (c) a nucleic acid sequence that forms a stem loop structure that can interact with the engineered DNA-dependent DNA polymerase of the invention.

A seventh aspect of the invention provides a method of modifying a target nucleic acid, the method comprising contacting the target nucleic acid with: the first complex of the invention, thereby modifying the target nucleic acid.

An eighth aspect of the invention provides a method of modifying a target nucleic acid, the method comprising contacting the target nucleic acid with: (a) a first sequence-specific DNA binding protein that is capable of binding to a first site on a target nucleic acid; (b) a first DNA-dependent DNA polymerase; (c) a first DNA endonuclease; and (d) a first DNA encoded repair template, thereby modifying the target nucleic acid.

A ninth aspect of the invention provides a method of modifying a target nucleic acid, the method comprising contacting the target nucleic acid with: (a) a first sequence-specific DNA binding protein that is capable of binding to a first site on a target nucleic acid and comprises nickase activity and/or endonuclease activity that is capable of introducing a single stranded nick or a double strand break; (b) a first DNA-dependent DNA polymerase; and (c) a first DNA encoded repair template, thereby modifying the target nucleic acid.

A tenth aspect of the invention provides a system for modifying a target nucleic acid comprising the first complex of the invention, a polynucleotide encoding the same, and/or the expression cassette or vector comprising the polynucleotide, wherein (a) the first sequence-specific DNA binding protein comprising DNA endonuclease activity binds to a first site on the target nucleic acid; (b) the first DNA-dependent DNA polymerase is capable of interacting with the first sequence-specific DNA binding protein and is recruited to the first sequence specific DNA binding protein and to the first site on the target nucleic acid, and (c) (i) the first DNA encoded repair template is linked to a first guide nucleic acid that comprises a spacer sequence having substantial complementarity to the first site on the target nucleic acid, thereby guiding the first DNA encoded repair template to the first site on the target nucleic acid, or (c)(ii) the first DNA encoded repair template is capable of interacting with the first sequence- specific DNA binding protein or the first DNA-dependent DNA polymerase and is recruited to the first sequence-specific DNA binding protein or the first DNA-dependent DNA polymerase and to the first site on the target nucleic acid, thereby modifying the target nucleic acid.

An eleventh aspect of the invention provides a system for modifying a target nucleic acid comprising the first complex of the invention, a polynucleotide encoding the same, and/or the expression cassette or vector comprising the polynucleotide, wherein (a) the first sequence-specific DNA binding protein binds to a first site on the target nucleic acid, (b) the first DNA endonuclease is capable of interacting with the first sequence specific DNA binding protein and/or a guide nucleic acid and is recruited to the first sequence specific DNA binding protein and to the first site on the target nucleic acid; (c) the first DNA-dependent DNA polymerase is capable of interacting with the first sequence specific DNA binding protein and/or a guide nucleic acid and is recruited to the first sequence specific DNA binding protein and to the first site on the target nucleic acid; and (d) (i) the first DNA encoded repair template is linked to a guide nucleic acid that comprises a spacer sequence having substantial complementarity to the first site on the target nucleic acid, thereby guiding the first DNA encoded repair template to the first site on the target nucleic acid, or (d)(ii) the first DNA encoded repair template is capable of interacting with the first sequence-specific DNA binding protein or the first DNA-dependent DNA polymerase and is recruited to the sequence-specific DNA binding protein or the first DNA-dependent DNA polymerase and to the first site on the target nucleic acid, thereby modifying the target nucleic acid.

### BRIEF DESCRIPTION OF THE SEQUENCES

SEQ ID NOs:1-20 are example Cas12a amino acid sequences useful with this invention.

SEQ ID NO:21-22 are exemplary regulatory sequences encoding a promoter and intron.

SEQ ID NOs:23-25 provide example peptide tags and corresponding affinity polypeptides.

SEQ ID NO:26-36 provide example RNA recruiting motifs and corresponding affinity polypeptides.

SEQ ID NOs:37-39 provide examples of a protospacer adjacent motif position for a Type V CRISPR-Cas12a nuclease.

SEQ ID NOs:40-47 provide example HUH-tags and corresponding recognition sequences.

SEQ ID NOs:48-58 and 88-94 provide example DNA-dependent DNA polymerases from various different organisms.

SEQ ID NOs:59-62 provide example Cas9 sequences.

SEQ ID NOs:63-70 provide example retron reverse transcriptases and retron scaffolds.

SEQ ID NOs:71-74 provide example chimeric guide nucleic acid sequences.

SEQ ID NO:75 provides an example Cas12a ribonucleoprotein (RNP).

SEQ ID NOs:76-87 provides the target sequence and crRNA sequences from Example 11.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the reference organism. A “homologous” nucleic acid sequence is a nucleotide sequence naturally associated with a host cell into which it is introduced.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

A “portion” or “fragment” of a nucleotide sequence of the invention will be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of and/or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the invention may be, where appropriate, included in a larger polynucleotide of which it is a constituent. As an example, a repeat sequence of guide nucleic acid of this invention may comprise a portion of a wild type CRISPR-Cas repeat sequence (e.g., a wild Type CRISR-Cas repeat; e.g., a repeat from the CRISPR Cas system of a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like).

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCGR Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX® version 2.0 for translated nucleotide sequences and BLASTN® version 2.0 for polynucleotide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2× SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1× SSC at 45°° C. for 15 minutes. An example of a low stringency wash for a duplex of, e.g., more than 100 nucleotides, is 4-6× SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

Any polynucleotide, nucleic acid construct, expression cassette and/or vector of this invention may be codon optimized for expression in any species of interest. Codon optimization is well known in the art and involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. As is understood in the art, codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function (and in some embodiments, the same structure) as that encoded by the original nucleotide sequence. Thus, in some embodiments, the polynucleotides, nucleic acid constructs, expression cassettes, and/or vectors of the invention (e.g., comprising/encoding a sequence specific DNA binding domain, a DNA-dependent DNA polymerase, a DNA endonuclease, and the like) may be codon optimized for expression in an organism (e.g., a plant (e.g., in a particular plant species), an animal, a bacterium, a fungus, etc.). In some embodiments, the codon optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the polynucleotides, nucleic acid constructs, expression cassettes, and/or vectors of the invention that have not been codon optimized.

In any of the embodiments described herein, a polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a DNA binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid, or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, a promoter region may comprise at least one intron (e.g., SEQ ID NOs:21 or 22).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12:619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP U.S. Pat. No. 255,378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9 (5): 297-306 (2015)), ZmSTK2_USP from maize (Wang et al. Genome 60 (6): 485-495 (2017)), LAT52 and LAT59 from tomato (Twell et al. Development 109 (3): 705-713 (1990)), Zm13 (U.S. Pat. No. 10,421,972), PLA₂-δ promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8): 1108-1115), corn light harvesting complex promoter (Bansal et al. (1992) Proc. Natl. Acad. Sci. USA 89:3654-3658), corn heat shock protein promoter (O′Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a nucleic acid construct of the invention (e.g., a sequence specific DNA binding polypeptide or domain, a DNA-dependent DNA polymerase (e.g., engineered DNA-dependent DNA polymerase), a DNA endonuclease polypeptide or domain, a DNA encoded repair template, a guide nucleic acid, a first complex, second complex, third complex, etc.), wherein the nucleic acid construct is operably associated with one or more control sequences (e.g., a promoter, terminator and the like). Thus, some embodiments of the invention provide expression cassettes designed to express, for example, one or more polynucleotides of the invention. When an expression cassette comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter, or they may be different promoters. Thus, for example, a polynucleotide encoding a sequence specific DNA binding polypeptide or domain, a polynucleotide encoding a DNA endonuclease polypeptide or domain, a polynucleotide encoding a DNA-dependent DNA polymerase polypeptide or domain, a DNA encoded repair template and/or a guide nucleic acid when comprised in an expression cassette may each be operably linked to a separate promoter or they may be operably linked to two or more promoters in any combination. In some embodiments, an expression cassette and/or the polynucleotides comprised therein in may be optimized for expression in a plant.

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to a gene encoding a sequence specific DNA binding polypeptide, a gene encoding a DNA endonuclease polypeptide, a gene encoding a DNA-dependent DNA polymerase, and the like, may be native to a host cell, or may be native to another source (e.g., foreign or heterologous to the promoter, to a gene encoding a sequence specific DNA binding polypeptide, a gene encoding a DNA endonuclease polypeptide, a gene encoding a DNA-dependent DNA polymerase, and the like, to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered, or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid construct or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As a non-limiting example, a target nucleic acid may be contacted with a sequence specific DNA binding domain, a DNA endonuclease, a DNA-dependent DNA polymerase, a DNA encoded repair template, a guide nucleic acid and/or a nucleic acid construct/expression cassette encoding/comprising the same, under conditions whereby the sequence specific DNA binding protein, DNA endonuclease, and the DNA-dependent DNA polymerase are expressed and the sequence specific DNA binding protein binds to the target nucleic acid, and the DNA-dependent DNA polymerase is either fused to the sequence specific DNA binding protein or is recruited to the sequence specific DNA binding protein (e.g., via a peptide tag fused to the sequence specific DNA binding protein and an affinity polypeptide (e.g., a polypeptide capable of binding the peptide tag) fused to the DNA-dependent DNA polymerase), thereby recruiting the DNA-dependent DNA polymerase to the vicinity of the target nucleic acid), thereby modifying the target nucleic acid.

As used herein, “modifying” or “modification” in reference to a target nucleic acid includes editing (e.g., mutating), covalent modification, exchanging/substituting nucleic acids/nucleotide bases, deleting, cleaving, nicking, and/or transcriptional control of a target nucleic acid. In some embodiments, a modification may include an indel of any size and/or a single base change (SNP) of any type.

“Introducing,” “introduce,” “introduced” (and grammatical variations thereof) in the context of a polynucleotide of interest means presenting a nucleotide sequence of interest (e.g., polynucleotide, a nucleic acid construct, and/or a guide nucleic acid) to a host organism or cell of said organism (e.g., host cell; e.g., a plant cell, animal cell, bacterial cell, fungal cell) in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes encoding, for example, a sequence specific DNA binding polypeptide or domain, a DNA endonuclease polypeptide or domain, a DNA-dependent DNA polymerase polypeptide or domain, etc.) may be transiently introduced into a cell with a guide nucleic acid and as such, no DNA maintained in the cell.

A nucleic acid construct of the invention can be introduced into a cell by any method known to those of skill in the art. In some embodiments of the invention, transformation of a cell comprises nuclear transformation. In other embodiments, transformation of a cell comprises plastid transformation (e.g., chloroplast transformation). In still further embodiments, the recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)).

A nucleotide sequence therefore can be introduced into a host organism or its cell in any number of ways that are well known in the art. The methods of the invention do not depend on a particular method for introducing one or more nucleotide sequences into the organism, only that they gain access to the interior of at least one cell of the organism. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, and/or in separate transformation events, or, alternatively, where relevant, a nucleotide sequence can be incorporated into a plant, for example, as part of a breeding protocol.

Endogenous DSB repair through homologous recombination is difficult to manipulate and faces competition from error-prone non-homologous end joining pathways. In this invention, templated editing is improved bypassing steps of DSB that reduce the efficiency of repair. Utilizing novel combinations of polypeptides and nucleic acids and protein-protein fusion and non-covalent recruitments, we deliver high fidelity, processive or distributive DNA polymerases and a repair template, sequence-specifically to the target site, which is cleaved or nicked by either a sequence specific DNA binding domain that comprises DNA endonuclease or nickase activity or by a DNA endonuclease having endonuclease or nickase activity, which is provided in combination with the sequence specific DNA binding protein. Using the DNA encoded repair template and the target DNA having a single stranded nick or a double strand break as primer, the DNA dependent DNA polymerase can initiate DNA synthesis immediately and copy the desired mutation or large insertion fragment into the target site. This invention can be used to generate specific changes of a single or a few bases, deletion of defined genome sequence, or insertion of small or large fragments.

Multiple DNA recruitment strategies may be used as described herein for improving delivery of a repair template to the target including, for example, HUH-tag, DNA aptamer, msDNA of bacterial retron and/or T-DNA recruitment. One specific example for improving template availability is the use of PCV, a type of HUH-tag. PCV domain can be, for example, fused to a CRISPR-Cas effector protein having nickase or endonuclease activity, which creates nick or break in target nucleic acid. A sequence of PCV recognition site is included in the repair template, so the repair template can be recruited to the target site through its ability to interact with the corresponding PCV domain. The recruitment can occur at roughly the same time a nick or break is created in the target nucleic acid by the CRISPR-Cas effector protein.

DNA-dependent DNA polymerase is an important component for carrying out homologous recombination. The 3′ end of target nucleic acid comprising a single stranded nick or a double strand break can anneal to a DNA encoded repair template and serve as a primer for DNA-dependent DNA polymerase to initiate strand synthesis, thereby copying genetic information from the repair template to the target site. In some embodiments, a DNA polymerase for use in this process may have high fidelity to prevent errors, and/or may exhibit high processivity to ensure long template being copied before DNA polymerase dissociates. In the context of association of a DNA-dependent DNA polymerase with a CRISPR-Cas effector polypeptide/complex that binds the target nucleic acid, it may be advantageous to have a DNA-dependent DNA polymerase with a distributive functionality to maximize the efficiency of template incorporation into the target. To accelerate this step, a DNA-dependent DNA polymerase with high fidelity plus processive and distributive profiles can be recruited either by protein fusion or non-covalent interaction with, for example, a sequence-specific DNA binding domain and DNA endonuclease (e.g., a CRISPR-Cas effector protein). Direct fusion can be done via optimized linker architecture. Non-covalent recruitment strategies can include recruitment via a guide nucleic acid (e.g., an RNA recruiting motif, e.g., MS2 loop) or recruitment via a sequence specific DNA binding domain (e.g., a CRISPR-Cas effector protein) and/or DNA endonuclease (e.g., via a peptide tag, e.g., antibody/epitope interaction, e.g., SunTag). Of course, the invention is not limited by these specific recruitment techniques and any other known or later developed protein-protein or nucleic acid-protein recruitment techniques now known or later developed may be used to carry out this invention.

The present inventors have developed compositions and methods that provide improved templated editing. Using combinations of protein-protein fusion and non-covalent recruitments, high fidelity, processive or distributive DNA polymerases are delivered sequence-specifically to a target site, which site may be cleaved or nicked by, for example, a CRISPR endonuclease or nickase. The DNA dependent DNA polymerase can initiate DNA synthesis immediately and copy the desired mutation or large insertion fragment into the target site by using the target DNA having a single stranded nick or a double strand break as a primer in combination with a DNA encoded repair template. The invention described herein and variations thereof can be utilized to make specific changes of a single or a few bases, deletion of defined genome sequence, or insertion of small or large fragments.

Thus, in some embodiments, the present invention provides a complex (e.g., a first complex) comprising: (a) a sequence-specific DNA binding protein (e.g., a first sequence-specific DNA binding protein) that is capable of binding to a site (e.g., a first site) on a target nucleic acid; and (b) a DNA-dependent DNA polymerase (e.g., a first DNA-dependent DNA polymerase). In some embodiments, the complex may comprise a DNA encoded repair template (e.g., a first DNA encoded repair template). In some embodiments, the complex may comprise a DNA endonuclease (e.g., a first DNA endonuclease), wherein the DNA endonuclease is capable of introducing a single stranded nick or a double strand break or wherein the sequence-specific DNA binding protein that is capable of binding to the site (e.g., the first site) on a target nucleic acid also comprises endonuclease activity that is capable of introducing a single stranded nick or a double strand break (e.g., a CRISPR-Cas effector protein).

In some embodiments, the present invention provides a complex (e.g., a first complex) comprising: (a) a sequence-specific DNA binding protein (e.g., a first sequence-specific DNA binding protein) that is capable of binding to a site (e.g., a first site) on a target nucleic acid and comprises endonuclease activity that is capable of introducing a single stranded nick or a double strand break; (b) a first DNA-dependent DNA polymerase; and (c) a DNA encoded repair template (e.g., a first DNA encoded repair template).

In some embodiments, the present invention provides a complex (e.g., a first complex) comprising: (a) a sequence-specific DNA binding protein (e.g., a first sequence-specific DNA binding protein) that is capable of binding to a site (e.g., a first site) on a target nucleic acid; (b) a DNA-dependent DNA polymerase (e.g., a first DNA-dependent DNA polymerase); (c) a DNA endonuclease (e.g., a first DNA endonuclease); and (d) a DNA encoded repair template (e.g., a first DNA encoded repair template).

In some embodiments, a sequence-specific DNA binding protein of a complex (e.g., a first complex) of the invention may be from a polynucleotide-guided endonuclease, a CRISPR-Cas effector protein, a protein-guided endonuclease (e.g., a zinc finger nuclease), a transcription activator-like effector nuclease (TALEN) and/or an Argonaute protein. In some embodiments, a sequence-specific DNA binding protein may be from a CRISPR-Cas polypeptide, a zinc finger, a transcription activator-like effector and/or an Argonaute protein.

In some embodiments, a DNA endonuclease or DNA endonuclease activity useful with a complex (e.g., a first complex) of the present invention may be or be from an endonuclease (e.g., Fok1), a polynucleotide-guided endonuclease, a CRISPR-Cas effector protein, a protein-guided endonuclease (e.g., a zinc finger nuclease), and/or a transcription activator-like effector nuclease (TALEN). In some embodiments, a DNA endonuclease may be a nuclease or a nickase or a DNA endonuclease activity may be a nuclease activity or a nickase activity.

In some embodiments, a sequence-specific DNA binding protein may be fused to a DNA-dependent DNA polymerase, optionally via a linker. In some embodiments, a sequence-specific DNA binding protein may be fused at its N-terminus to a DNA-dependent DNA polymerase. In some embodiments, a sequence-specific DNA binding protein may be fused at its C-terminus to a DNA-dependent DNA polymerase.

The present invention further provides an engineered (modified) DNA-dependent DNA polymerase fused to an affinity polypeptide that is capable of interacting with a peptide tag or an RNA recruiting motif. In some embodiments, an engineered DNA-dependent DNA polymerase of the invention may comprise a DNA-dependent DNA polymerase fused to a sequence non-specific DNA binding domain, optionally wherein the sequence non-specific DNA binding domain may be a sequence-nonspecific dsDNA binding protein from Sso7d from Sulfolobus solfataricus. An engineered DNA-dependent DNA polymerase of the invention may exhibit increased processivity, increased fidelity, increased affinity, increased sequence specificity, decreased sequence specificity and/or increased cooperativity as compared to the same DNA-dependent DNA polymerase that is not engineered as described herein. In some embodiments, the engineered DNA-dependent DNA polymerase may be modified to reduce or eliminate at least one of 5′→3′-polymerase activity, 3′→5′ exonuclease activity, 5′→3′ exonuclease activity, and/or 5′→3′ RNA-dependent DNA polymerase activity. Thus an engineered DNA-dependent DNA polymerase may not comprise at least one activity of 5′→3′-polymerase activity, 3′→5′ exonuclease activity, 5′→3′ exonuclease activity, and/or 5′→3′ RNA-dependent DNA polymerase activity

In some embodiments, a sequence-specific DNA binding protein (e.g., a first sequence-specific DNA binding protein) may be fused to a peptide tag and a DNA-dependent DNA polymerase (e.g., a first DNA-dependent DNA polymerase) may be fused to an affinity polypeptide that is capable of binding the peptide tag, wherein the DNA-dependent DNA polymerase may be recruited to the sequence-specific DNA binding protein that is fused to the peptide tag (and to a target nucleic acid to which the sequence-specific DNA binding protein may be bound). In some embodiments, a DNA-dependent DNA polymerase (e.g., a first sequence-specific DNA binding protein) may be fused to a peptide tag and a sequence-specific DNA binding protein (e.g., a first sequence-specific DNA binding protein) may be fused to an affinity polypeptide that is capable of binding the peptide tag, thereby recruiting the DNA-dependent DNA polymerase to the sequence-specific DNA binding protein that is fused to the affinity polypeptide and to a target nucleic acid to which the sequence-specific DNA binding protein is bound.

A complex of the invention may further comprise a guide nucleic acid (e.g., a CRISPR nucleic acid, crRNA, crDNA). A guide nucleic acid may be used in combination with a CRISPR-Cas effector protein, which, in some embodiments, may comprise endonuclease activity or nickase activity. In some embodiments, endonuclease or nickase activity of a sequence-specific DNA binding protein may be from, for example, a polynucleotide-guided endonuclease, a CRISPR-Cas effector protein, a protein-guided endonuclease (e.g., a zinc finger nuclease), and/or a transcription activator-like effector nuclease (TALEN).

In some embodiments, a guide nucleic acid may be linked to a RNA-recruiting motif and a DNA-dependent DNA polymerase may be fused to an affinity polypeptide that is capable of binding the RNA recruiting motif. In some embodiments, an RNA recruiting motif may be linked to the 5′ end or to the 3′ end of the CRISPR nucleic acid (e.g., a recruiting crRNA, a recruiting crDNA).

In some embodiments, a DNA encoded repair template may be recruited to a target nucleic acid by linking the DNA encoded repair template to a guide nucleic acid that comprises a spacer having complementarity to the target nucleic acid.

The present invention may provide a further complex (e.g., a second complex), the complex comprising: (a) a sequence-specific DNA binding protein (e.g., a second sequence-specific DNA binding protein) that is capable of binding to a second site on a target nucleic acid; and (b) a DNA-encoded repair template (e.g., a first or a second DNA-encoded repair template). In some embodiments, the sequence-specific DNA binding protein may be from a polynucleotide-guided endonuclease, a CRISPR-Cas effector protein, a protein-guided endonuclease (e.g., a zinc finger nuclease), a transcription activator-like effector nuclease (TALEN) and/or an Argonaute protein. In some embodiments, the complex (e.g., second complex) may further comprise a DNA endonuclease (e.g., a second DNA endonuclease), wherein the DNA endonuclease is capable of introducing a single stranded nick or a double strand break into a target nucleic acid. In some embodiments, the DNA endonuclease may be from polynucleotide-guided endonuclease, a CRISPR-Cas effector protein, a protein-guided endonuclease (e.g., a zinc finger nuclease), a transcription activator-like effector nuclease (TALEN). In some embodiments, a sequence-specific DNA binding protein of the second complex that is capable of binding to a second site on a target nucleic acid may further comprise endonuclease activity that is capable of introducing a single stranded nick or a double strand break in a target nucleic acid. In some embodiments, the sequence-specific DNA binding protein (e.g., the second sequence-specific DNA binding protein) that further comprises endonuclease activity may be a polynucleotide-guided endonuclease, a CRISPR-Cas effector protein, a protein-guided endonuclease (e.g., a zinc finger nuclease), or a transcription activator-like effector nuclease (TALEN)).

In some embodiments, a DNA-encoded repair template may be linked to a DNA recruiting motif and the sequence-specific DNA binding protein may be fused to an affinity polypeptide that is capable of interacting with the DNA recruiting motif, optionally wherein the DNA recruiting motif/affinity polypeptide comprises a HUH-tag, DNA aptamer, msDNA of bacterial retron or antibody/epitope pair (e.g., T-DNA recruitment). In some embodiments, a sequence-specific DNA binding protein may be fused to a Porcine Circovirus 2 (PCV) Rep protein and the DNA template comprises a PCV recognition site. In some embodiments, a sequence-specific DNA binding protein may be fused at its N-terminus to the PCV Rep protein. In some embodiments, a sequence-specific DNA binding protein may be fused at its C-terminus to the PCV Rep protein. Non-limiting examples of HUH-Tags and their corresponding recognitions sequences that may be useful with this invention are provided in Table 1.

TABLE 1
HUH-Tags and recognitions sequences

| HUH-tag | recognition sequence |
|---|---|
| porcine circovirus 2 | AAGTATTACCAGAAA |
| Rep protein | SEQ ID NO: 40 |
| duck circovirus Rep | AAGTATTACCAGAAA |
| protein | SEQ ID NO: 41 |
| fava bean necrosis | AAGTATTACCAGAAA |
| yellow virus Rep | SEQ ID NO: 42 |
| protein | |
| RepB Streptococcus | TGCTTCCGTACTACGACCCCCCA |
| agalactiae | SEQ ID NO: 43 |
| RepB Fructobacillus | TGCTTCCGTACTACGACCCCCCA |
| tropaeoli | SEQ ID NO: 44 |
| conjugation protein | TTTGCGTGGGGTGTGGTGCTTT |
| TraI Escherichia coli | SEQ ID NO: 45 |
| mobilization protein | CCAGTTTCTCGAAGAGAAACCGG |
| A Escherichia coli | TAAGTGCACCCTCCC |
| | SEQ ID NO: 46 |
| nicking enzyme | ACGCGAACGGAACGTTCGCATAA |
| Staphylococcus | GTGCGCCCTTACGGGATTTAAC |
| aureus | SEQ ID NO: 47 |

In some embodiments, a DNA encoded repair template may be recruited to a target nucleic acid by integrating the DNA encoded repair template into a T-DNA sequence that interacts with an Agrobacterium effector protein (e.g., an Agrobacterium virulence polypeptide, optionally, virD2 and/or virE2), wherein the sequence specific DNA binding protein, for example, may be recruited to the Agrobacterium effector protein, thereby recruiting the DNA encoded repair template to the sequence specific DNA binding protein and to the target nucleic acid that the sequence specific DNA binding protein binds. As an example, one or more epitope tags may be fused to the sequence specific DNA binding protein and an antibody that recognizes the epitope tag(s) may be fused to the Agrobacterium effector protein, thereby enabling the sequence specific DNA binding protein and the Agrobacterium effector protein to interact in the plant cell. Any T-DNA sequence associated with the Agrobacterium effector protein would be recruited to the target nucleic acid by the action of the sequence specific DNA binding protein.

In some embodiments, a DNA encoded repair template may be recruited to a target nucleic acid by attachment of a DNA aptamer to the DNA encoded repair template. A DNA aptamer is a sequence of DNA that can bind to a specific target with high affinity due to its unique secondary structure. DNA aptamer guided gene targeting has been demonstrated for endonuclease I-SceI mediated gene targeting in human and yeast system. A pool of candidate DNA aptamers may be screened by capillary electrophoresis for affinity with specific CRIPSR protein (Cas9, Cpf1, etc). DNA aptamers with the highest affinity to the selected CRISPR nuclease protein will be attached to single strand DNA template to guide the DNA template to the CRISPR protein target locus.

In some embodiments, the repair template can be expressed as msDNA from a bacterial retron scaffold attached to guide RNA. Bacterial retrons are bacterial elements that encode a reverse transcriptase which recognize a specific part of transcribed retron genome and use it as template to produce multiple copies of single strand DNA (msDNA). The msDNA remains tethered to the RNA template. A retron RNA scaffold sequence can be added to CRISPR guide RNA scaffold as an extension with part of retron genome replaced with desired repair template for gene editing. Expression of the template as an msDNA tethered to the guide RNA scaffold extension enables delivery of multiple copies of repair template to break sites at the same time the break been made. This system has been demonstrated in yeast, but not in mammalian or plant systems. Exemplary bacterial retrons useful with this invention are provided in Table 2.

TABLE 2
Examples of bacterial retrons

| Retron | Retron Reverse Transcriptase | Retron scaffold |
|---|---|---|
| ec67 | atgacaaaaacatctaaacttgacgcacttagggctgctacttcacgtgaagacttgg | cacgcatgtaggcagatttgttg |
| | ctaaaattttagatattaagttggtatttttaactaacgttctatatagaatcggctc | gttgtgaatcgcaaccagtggcc |
| | ggataatcaatacactcaatttacaataccgaagaaaggaaaaggggtaaggactatt | ttaatggcaggaggaatcgcctc |
| | tctgcacctacagaccggttgaaggacatccaacgaagaatatgtgacttactttctg | cctaaaatccttgattcagagct |
| | attgtagagatgagatctttgctataaggaaaattagtaacaactattcctttggttt | atacggcaggtgtgctgtgcgaa |
| | tgagaggggaaaatcaataatcctaaatgcttataagcatagaggcaaacaaataata | ggagtgcctgcatgcgt |
| | ttaaatatagatcttaaggatttttttgaaagctttaattttggacgagttagaggat | SEQ ID NO: 64 |
| | attttctttccaatcaggattttttattaaatcctgtggtggcaacgacacttgcaaa | |
| | agctgcatgctataatggaaccctcccccaaggaagtccatgttctcctattatctca | |
| | aatctaatttgcaatattatggatatgagattagctaagctggctaaaaaatatggat | |
| | gtacttatagcagatatgctgatgatataacaatttctacaaataaaaatacatttcc | |
| | gttagaaatggctactgtgcaacctgaaggggttgttttgggaaaagttttggtaaaa | |
| | gaaatagaaaactctggattcgaaataaatgattcaaagactaggcttacgtataaga | |
| | catcaaggcaagaagtaacgggacttacagttaacagaatcgttaatattgatagatg | |
| | ttattataaaaaaactcgggcgttggcacatgctttgtatcgtacaggtgaatataaa | |
| | gtgccagatgaaaatggtgttttagtttcaggaggtctggataaacttgaggggatgt | |
| | ttggttttattgatcaagttgataagtttaacaatataaagaaaaaactgaacaagca | |
| | acctgatagatatgtattgactaatgcgactttgcatggttttaaattaaagttgaat | |
| | gcgcgagaaaaagcatatagtaaatttatttactataaattttttcatggcaacacct | |
| | gtcctacgataattacagaagggaagactgatcggatatatttgaaggctgctttgca | |
| | ttctttggagacatcatatcctgagttgtttagagaaaaaacagatagtaaaaagaaa | |
| | gaaataaatcttaatatatttaaatctaatgaaaagaccaaatattttttagatcttt | |
| | ctgggggaactgcagatctgaaaaaatttgtagagcgttataaaaataattatgcttc | |
| | ttattatggttctgttccaaaacagccagtgattatggttcttgataatgatacaggt | |
| | ccaagcgatttacttaattttctgcgcaataaagttaaaagctgcccagacgatgtaa | |
| | ctgaaatgagaaagatgaaatatattcatgttttctataatttatatatagttctcac | |
| | accattgagtccttccggcgaacaaacttcaatggaggatcttttccctaaagatatt | |
| | ttagatatcaagattgatggtaagaaattcaacaaaaataatgatggagactcaaaaa | |
| | cggaatatgggaagcatattttttccatgagggttgttagagataaaaagcggaaaat | |
| | agattttaaggcattttgttgtatttttgatgctataaaagatataaaggaacattat | |
| | aaattaatgttaaatagctaa SEQ ID NO: 63 | |
| ec86 | atgaaatcgcatgatcgattgaggatcgtctttgctcagatccgccagaactggcgg | Atgcgcacccttagcgagaggtt |
| | cttttgctcatgttatgcatgtgcatgaaaaccactgcataa | tatcattaaggtcaacctctgga |
| | SEQ ID NO: 65 | tgttgtttcggcatcctgcattg |
| | | aatctgagttactgtctgttttc |
| | | cttgttggaacggagagcatcgc |
| | | ctgatgctctccgagccaaccag |
| | | gaaacccgttttttctgacgtaa |
| | | gggtgcgca SEQ ID NO: 66 |
| ec107 | Atggatgctacccggacaacccttctggcgctcgatttgttcggctcgccgggctgga | Cgccagcagtggcaatagcgttt |
| | gcgccgataaagaaatacagcgactgcatgcgctcagtaatcatgccggacgccatta | ccggccttttgtgccgggagggt |
| | ccgacgcattattctttctaaacgccacggtggtcagcggctggtgttagcccctgat | cggcgagtcgctgacttaacgcc |
| | tacttgctcaaaaccgtacagcgcaacattcttaagaacgtcctttcacaatttccgc | agtagtatgtccatatacccaaa |
| | tttccccttttgctacagcctaccgaccaggttgcccaatcgtcagcaacgcgcagcc | gtcgcttcattgtacctgagtac |
| | acactgccaacagccgcagatcctgaaactcgatatcgaaaactttttcgatagcatt | gcttcgcgtacgtcgcgctgacg |
| | agctggttacaggtctggcgtgtgtttcgccaggcccagttgccacgtaatgtggtaa | cgctcagtacagttacgcgcctt |
| | ccatgctgacctggatttgttgttataacgacgcgttaccgcagggggcaccaacttc | cgggatggtttaatg |
| | gccagccatttccaatcttgtgatgcgccgttttgatgaacgcataggggaatggtgt | SEQ ID NO: 68 |
| | caggctcggggaattacctacacccgctactgcgatgacatgaccttttcaggtcact | |
| | tcaatgcccgccaggttaaaaataaagtgtgcggattgttagcggagctgggcctgag | |
| | cctcaataaacgcaaaggctgcctgatagctgcctgtaagcgccagcaagtaaccggg | |
| | attgttgttaatcacaagccacagcttgcccgtgaagcgcgccgggcgctgcgtcagg | |
| | aggtgcatttgtgccaaaaatatggcgttatttcgcatcttagtcatcgtggtgaact | |
| | tgatccttctggcgatctccacgcacaggcaacggcgtatctttatgctttgcaggga | |
| | agaataaactggttattgcaaatcaaccctgaggatgaggcctttcaacaggcgagag | |
| | agagtgtaaagcgaatgctggttgcatggtaa SEQ ID NO: 67 | |
| mx162 | atgaccgccaggctggacccgttcgtccccgcagcttcgccgcaggccgtgcccacgc | agaggtccggagtgcatcagcct |
| | ccgagctcaccgctccgtcgtcagacgcggccgcgaagcgtgaagcccgccggctcgc | gagcgcctcgagcggcggagcgg |
| | gcacgaagcgttgctcgtccgcgcgaaggccatcgacgaagcgggcggcgccgacgac | cgttgcgccgctccggttggaat |
| | tgggtgcaggcgcagctcgtctccaagggcctcgcggtggaggacctggacttctcca | gcaggacactctccgcaaggtag |
| | gcgcctccgagaaggacaagaaggcctggaaggagaagaagaaggccgaggccaccga | cctgttcttggctctctccctcc |
| | gcgccgcgcgctgaagcgtcaggcgcacgaggcgtggaaggccacgcacgtgggccac | taggcactacggccagggtgggt |
| | ctgggcgcgggcgtgcactgggcggaggaccgcctggccgacgcgttcgacgtgcccc | agcggagccaacgacgcgaccgc |
| | accgcgaggagcgcgcccgggccaacggcctgacggagctggactcggcggaggcgct | cgtttacccaccccggccgtagt |
| | ggccaaggcgctggggctgagcgtgtccaagctgcgctggttcgcgttccaccgcgag | gcctaggaggggagagccggtga |
| | gtggacacggccacgcactacgtgagctggacgattccgaagcgggacggcagcaagc | ggctaccgtgccccaggtaagatg |
| | gcacgattacgtcccccaagcctgagctgaaggcagcgcagcgctgggtgctgtccaa | SEQ ID NO: 70 |
| | cgtcgtggagcggctgccggtgcacggcgcggcgcacggcttcgtggcgggacgctcc | |
| | atcctcaccaacgcgctggcccaccagggcgcggacgtggtggtgaaggtggacctca | |
| | aggacttcttcccctccgtcacctggcgccgggtgaagggcctgttgcgcaagggcgg | |
| | cctgcgggagggcacgtccacgctgctgtcgctgctctccacggaagcgccgcgggag | |
| | gcggtgcagttccggggcaagctgctgcacgtggccaagggcccgcgcgcgctgcccc | |
| | agggcgcgcccacgtcgccgggcatcaccaacgcgctgtgcctgaagctggacaagcg | |
| | gctgtccgcgctcgcgaagcggctgggcttcacgtacacgcgctacgcggacgacctg | |
| | accttctcgtggacgaaggcgaagcagcccaagccgcggcggacgcagcgtcccccgg | |
| | tggcggtgctgctgtctcgcgtgcaggaagtggtggaggcggagggcttccgcgtgca | |
| | cccggacaagacgcgcgtggcgcgcaagggcacgcggcagcgggtgacggggctggtc | |
| | gtgaatgcggcgggcaaggacgcgccggcggcccgagtcccgcgcgacgtggtgcgcc | |
| | agctccgcgccgccatccacaaccggaagaagggcaagccgggccgcgagggcgagtc | |
| | gctggagcagctcaagggcatggccgccttcatccacatgacggacccggccaagggc | |
| | cgcgccttcctggctcagctcacggagctggagtccacggcgagcgcggctccgcagg | |
| | cggagtga SEQ ID NO: 69 | |

Examples of Chimeric guide nucleic acid sequence (guide DNA) designed to introduce templated editing in a human genome target FANCF01 include, but are not limited to:
Repair template (bold) in embed in ec67 retron scaffold followed by single guide nucleic acid (sg nucleic acid) (italic lower case):

SEQ ID NO: 71
CACGCATGTAGGCAGATTTGTTGGTTGTGAATCGCAACCAGTGGCCTTA
ATGGCAGGAGGAATCGCCTCCAGAGTCGCCGTCTCCAAGGTGAAAGCGG
AAGTAGGGCCTTCGCGCACCTCATGGAATCCCTTCTGCAGCACCTAGAT
CGCTTTTCTGAACTCCTAGCAGTATCTAGCACTACCTACGTCAGCACCT
GGGACCCCGCGGTGTGCTGTGCGAAGGAGTGCCTGCATGCGTggaatcc
cttctgcagcaccgttttagagctagaaatagcaagttaaaataaggct
agtccgttatcaacttgaaaaagtggcaccgagtcggtgc

Repair template (bold) in embed in ec86 retron scaffold followed by single guide nucleic acid (sg nucleic acid) (italic lower case):

SEQ ID NO: 72
ATGCGCACCCTTAGCGAGAGGTTTATCATTAAGGTCAACCTCTGGATGT
TGTTTCGGCATCCTGCATTGAATCTGAGTTACTGTCTGTTTTCCTAGAG
TCGCCGTCTCCAAGGTGAAAGCGGAAGTAGGGCCTTCGCGCACCTCATG
GAATCCCTTCTGCAGCACCTAGATCGCTTTTCTGAACTCCTAGCAGTAT
CTAGCACTACCTACGTCAGCACCTGGGACCCCGCCAGGAAACCCGTTTT
TTCTGACGTAAGGGTGCGCAggaatcccttctgcagcaccgttttagag
ctagaaatagcaagttaaaataaggctagtccgttatcaacttgaaaaa
gtggcaccgagtcggtgc

Repair template (bold) in embed in ec107 retron scaffold followed by single guide nucleic acid (sg nucleic acid) (italic lower case):

SEQ ID NO: 73
GCCAGCAGTGGCAATAGCGTTTCCGGCCTTTTGTGCCGGGAGGGTCGGC
GAGTCGCTGACTTAACGCCAGTAGTATGTCCATATACCCAAGAGTCGCC
GTCTCCAAGGTGAAAGCGGAAGTAGGGCCTTCGCGCACCTCATGGAATC
CCTTCTGCAGCACCTAGATCGCTTTTCTGAACTCCTAGCAGTATCTAGC
ACTACCTACGTCAGCACCTGGGACCCCGCGGGATGGTTTAATGGTATTG
CCGCggaatcccttctgcagcaccgttttagagctagaaatagcaagtt
aaaataaggctagtccgttatcaacttgaaaaagtggcaccgagtcggt
gc

Repair template (bold) in embed in mx162 retron scaffold followed by single guide nucleic acid (sg nucleic acid) (italic lower case):

SEQ ID NO: 74
AGAGGTCCGGAGTGCATCAGCCTGAGCGCCTCGAGCGGCGGAGCGGCGT
TGCGCCGCTCCGGTTGGAATGCAGGACACTCTCCGCAAGGTAGAGTCGC
CGTCTCCAAGGTGAAAGCGGAAGTAGGGCCTTCGCGCACCTCATGGAAT
CCCTTCTGCAGCACCTAGATCGCTTTTCTGAACTCCTAGCAGTATCTAG
CACTACCTACGTCAGCACCTGGGACCCCGCTGAGGCTACCGTGCCCCAG
GTAAGATGGTGGTGCTTTCCCGGggaatcccttctgcagcaccgtttta
gagctagaaatagcaagttaaaataaggctagtccgttatcaacttgaa
aaagtggcaccgagtcggtgc

In some embodiments, a complex (e.g., a second complex) may further comprise a DNA-dependent DNA polymerase (e.g., a second DNA-dependent DNA polymerase).

In some embodiments, a complex (e.g., a second complex) may further comprise a guide nucleic acid, optionally wherein the guide nucleic acid may be linked to the DNA-encoded repair template (e.g., a first or a second DNA-encoded repair template).

In some embodiments, a third complex may be provided, the third complex comprising a sequence-specific DNA binding protein (e.g., a third sequence-specific DNA binding protein) that is cable of binding to a site (e.g., a third site) on a target nucleic acid that is on a different strand from the first site and second and a DNA endonuclease (e.g., a third DNA endonuclease) (e.g., a nickase that can generate a single strand break). In some embodiments, contacting the target nucleic acid with the third complex may boost the efficiency of repair by improving mismatch repair.

In some embodiments, the invention provides an RNA molecule, the RNA molecule comprising (a) a nucleic acid sequence that mediates interaction with a CRISPR-Cas effector protein; (b) a nucleic acid sequence that directs the CRISPR-Cas effector protein to a specific nucleic acid target site through a DNA-RNA interaction, and (c) a nucleic acid sequence that forms a stem loop structure (e.g., an RNA recruiting motif) that can interact with the engineered DNA-dependent DNA polymerase of the present invention. In some aspects, the invention provides an engineered DNA-dependent DNA polymerase of the invention complexed with the RNA molecule comprising (a) a nucleic acid sequence that mediates interaction with a CRISPR-Cas effector protein; (b) a nucleic acid sequence that directs the CRISPR-Cas effector protein to a specific nucleic acid target site through a DNA-RNA interaction, and (c) a nucleic acid sequence that forms a stem loop structure.

The present invention further provides polynucleotides encoding the complexes of the invention (e.g., first complex, second complex, and/or third complex) and/or encoding one or more of the sequence-specific DNA binding proteins (e.g., the first sequence-specific DNA binding protein, second sequence-specific DNA binding protein, and/or third sequence-specific DNA binding protein), DNA-dependent DNA polymerases (e.g., the first DNA-dependent DNA polymerase and/or second DNA-dependent DNA polymerase), DNA endonucleases (e.g., the first DNA endonuclease, second DNA endonuclease, and/or third DNA endonuclease) or comprising one or more of the DNA encoded repair templates (e.g., the first DNA encoded repair template and/or second DNA encoded repair template) or one or more guide nucleic acids (e.g., a first guide nucleic acid, second guide nucleic acid, and/or third guide nucleic acid, and the like). In some embodiments, a polynucleotide encoding an engineered DNA-dependent DNA polymerase of the invention is provided. Further provided herein are one or more expression cassettes and/or vectors comprising one or more of the polynucleotides of the invention.

In some embodiments of the invention, polynucleotides encoding sequence specific DNA binding domains, sequence non-specific DNA binding proteins, DNA endonucleases, DNA-dependent DNA polymerases, and/or expression cassettes and/or vectors comprising the same may be codon optimized for expression in a cell or an organism (e.g., an organism and/or a cell of, for example, an animal (e.g., a mammal, an insect, a fish, and the like), a plant (e.g., a dicot plant, a monocot plant), a bacterium, an archaeon, and the like). In some embodiments, an expression cassette comprising the polynucleotides of the invention/encoding the complexes/polypeptides of the invention may be codon optimized for expression in a dicot plant or for expression in a monocot plant.

The present invention further provides methods of using the compositions of the invention for modifying target nucleic acids. Accordingly, the invention provides methods for modifying a target nucleic, the methods comprising contacting a target nucleic acid or a cell comprising the target nucleic acid with a complex or system of the invention, polynucleotides encoding/comprising the same, or one or more of the components of a complex or system of the invention, and/or expression cassettes and/or vectors comprising the same. The methods may be carried out in an in vivo system (e.g., in a cell or in an organism) or in an in vitro system (e.g., cell free). The polypeptides and complexes of the invention, and polynucleotides/expression cassettes/vectors encoding the same may be used in a method for modifying a target nucleic acid, for example, in a plant or plant cell, the method comprising introducing one or more expression cassettes of the invention into a plant or plant cell, thereby modifying the target nucleic acid in the plant or plant cell to produce a plant or plant cell comprising the modified target nucleic acid. In some embodiments, the method may further comprise regenerating the plant cell comprising the modified target nucleic acid to produce a plant comprising the modified target nucleic acid.

In some embodiments, a method of modifying a target nucleic acid is provided, the method comprising: contacting the target nucleic acid with a complex of the invention (e.g., a first complex), thereby modifying the target nucleic acid. In some embodiments, the method may further comprise contacting the target nucleic acid with a second complex of the invention, thereby modifying the target nucleic acid. In some embodiments, the target nucleic acid may be further contacted with a third complex of the invention, thereby improving the repair efficiency of the modifying of the target nucleic acid.

In some embodiments, a method of modifying a target nucleic acid is provided, the method comprising contacting the target nucleic acid with: (a) a first sequence-specific DNA binding protein that is capable of binding to a first site on a target nucleic acid; (b) a first DNA-dependent DNA polymerase; (c) a first DNA endonuclease; and (d) a first DNA encoded repair template, thereby modifying the target nucleic acid. In some embodiments, the first sequence-specific DNA binding protein, the first DNA-dependent DNA polymerase, the first DNA endonuclease, and the first DNA encoded repair template may form a complex, wherein the complex may interact with the target nucleic acid.

In some embodiments, a method of modifying a target nucleic acid is provided, the method comprising contacting the target nucleic acid with: (a) a first sequence-specific DNA binding protein that is capable of binding to a first site on a target nucleic acid, wherein the first sequence-specific DNA binding protein comprises nickase activity or endonuclease activity that can introduce a single stranded nick or a double strand break; (b) a first DNA-dependent DNA polymerase; and (c) a first DNA encoded repair template, thereby modifying the target nucleic acid. In some embodiments, the first sequence-specific DNA binding protein comprising endonuclease activity, the first DNA-dependent DNA polymerase, and the first DNA encoded repair template may form a complex that is capable of interacting with the target nucleic acid. The endonuclease activity and/or nickase activity of the first sequence-specific DNA binding protein may be from, for example, a polynucleotide-guided endonuclease, a CRISPR-Cas effector protein, a protein-guided endonuclease (e.g., a zinc finger nuclease), and/or a transcription activator-like effector nuclease (TALEN). In some embodiments, the first sequence-specific DNA binding protein comprising endonuclease activity may be a polynucleotide-guided endonuclease, a CRISPR-Cas effector protein, a protein-guided endonuclease (e.g., a zinc finger nuclease), and/or a transcription activator-like effector nuclease (TALEN). A first sequence-specific DNA binding protein may be, for example, from a polynucleotide-guided endonuclease, a CRISPR-Cas effector protein, a protein-guided endonuclease (e.g., a zinc finger nuclease), a transcription activator-like effector nuclease (TALEN) and/or an Argonaute protein.

In some embodiments, a first sequence-specific DNA binding protein may be fused to a first DNA-dependent DNA polymerase, optionally via a linker. In some embodiments, a first sequence-specific DNA binding protein may be fused at its N-terminus to a first DNA-dependent DNA polymerase. In some embodiments, a first sequence-specific DNA binding protein may be fused at its C-terminus to a first DNA-dependent DNA polymerase. In some embodiments, a first sequence-specific DNA binding protein may be fused to a peptide tag and a first DNA-dependent DNA polymerase may be fused to an affinity polypeptide that is capable of binding the peptide tag, thereby recruiting the first DNA-dependent DNA polymerase to the first sequence-specific DNA binding protein that is fused to the peptide tag and to a target nucleic acid to which the sequence-specific DNA binding protein is bound and/or is capable of binding. In some embodiments, a first DNA-dependent DNA polymerase may be fused to a peptide tag and a first sequence-specific DNA binding protein may be fused to an affinity polypeptide that is capable of binding the peptide tag, thereby recruiting the first DNA-dependent DNA polymerase to the first sequence-specific DNA binding protein fused to the affinity polypeptide and to a target nucleic acid to which the sequence-specific DNA binding protein is bound and/or is capable of binding.

In some embodiments of the invention, a first sequence-specific DNA binding domain and/or a first DNA endonuclease may be or may be from a CRISPR-Cas effector protein, wherein the target nucleic acid may be contacted with a guide nucleic acid (e.g., a CRISPR nucleic acid, crRNA, crDNA) (e.g., a first guide nucleic acid) that directs the CRISPR-Cas effector protein to a specific nucleic acid target site through a DNA-RNA interaction. In some embodiments, a DNA encoded repair template (e.g., a first DNA encoded repair template) may be linked to the guide nucleic acid, thereby guiding the DNA encoded repair template to the target nucleic acid. In some embodiments, a guide nucleic acid may be linked to a RNA-recruiting motif and a DNA-dependent DNA polymerase (e.g., a first DNA-dependent DNA polymerase) may be fused to an affinity polypeptide that is capable of binding the RNA recruiting motif, thereby guiding the DNA-dependent DNA polymerase to the target nucleic acid. An RNA recruiting motif may be linked to the 5′ end or to the 3′ end of the guide nucleic acid (e.g., a recruiting crRNA, a recruiting crDNA).

In some embodiments, the target nucleic acid contacted with the first complex of the invention may be contacted with a second complex of the invention, the second complex comprising: (a) a second sequence-specific DNA binding protein that is capable of binding to a second site on the target nucleic acid; and (b) a DNA-encoded repair template (e.g., a first DNA-encoded repair template or a second DNA-encoded repair template). In some embodiments, wherein the target nucleic acid is further contacted with a second DNA endonuclease or the second complex further comprises a second DNA endonuclease, wherein the second DNA endonuclease is capable of introducing into the target nucleic acid a single stranded nick or a double strand break. Alternatively, or in addition, the second sequence-specific DNA binding protein of the second complex may comprise endonuclease activity itself that may introduce a single stranded nick or a double strand break into the target nucleic acid. In some embodiments, the second sequence-specific DNA binding protein that is capable of binding to a second site on the target nucleic acid, the second DNA-encoded repair template, and optionally the DNA endonuclease may form a complex that interacts with the second site on the target nucleic acid.

In some embodiments, a second sequence-specific DNA binding protein may be fused to a peptide tag and a second DNA endonuclease may be fused to an affinity polypeptide that is capable of binding the peptide tag, thereby recruiting the second DNA endonuclease to the second sequence-specific DNA binding protein that is fused to the peptide tag and to the second site on the target nucleic acid to which the second sequence-specific DNA binding protein binds and/or is capable of binding. In some embodiments, a second DNA endonuclease may be fused to a peptide tag and a second sequence-specific DNA binding protein may be fused to an affinity polypeptide that is capable of binding the peptide tag, thereby recruiting second DNA endonuclease to the second sequence-specific DNA binding protein that is fused to the affinity polypeptide and to the second site on the target nucleic acid to which the second sequence-specific DNA binding protein binds and/or is capable of binding.

In some embodiments, a DNA-encoded repair template of a second complex (e.g., a first DNA-encoded repair template or a second DNA-encoded repair template) may be linked to a DNA recruiting motif and a second sequence-specific DNA binding protein may be fused to an affinity polypeptide that is capable of interacting with the DNA recruiting motif, optionally wherein the DNA recruiting motif/affinity polypeptide comprises a HUH-tag (see, e.g., Table 1), DNA aptamer, msDNA of bacterial retron or a T-DNA recruitment, thereby recruiting the second DNA-encoded repair template to the sequence-specific DNA binding protein and the target nucleic acid to which the sequence-specific DNA binding protein can bind. In some embodiments, a second sequence-specific DNA binding protein may be fused, for example, to a Porcine Circovirus 2 (PCV) Rep protein and the DNA encoded repair template may comprise a PCV recognition site.

In some embodiments, a second sequence-specific DNA binding protein may be from and/or may be a polynucleotide-guided endonuclease, a CRISPR-Cas effector protein, a protein-guided endonuclease (e.g., a zinc finger nuclease), a transcription activator-like effector nuclease (TALEN) and/or an Argonaute protein. In some embodiments, a second DNA binding domain and/or second DNA endonuclease may be from and/or may be a CRISPR-Cas effector protein, wherein the target nucleic acid may be contacted with a guide nucleic acid (e.g., a CRISPR nucleic acid, crRNA, crDNA) (e.g., a second guide nucleic acid) that directs the CRISPR-Cas effector protein to a specific nucleic acid target site through a DNA-RNA interaction. In some embodiments, a DNA encoded repair template (e.g., a second DNA encoded repair template) may be linked to the guide nucleic acid, thereby guiding the DNA encoded repair template to the target nucleic acid. In some embodiments, the second guide nucleic acid may be linked to an RNA-recruiting motif and a second DNA endonuclease may be fused to an affinity polypeptide that is capable of binding the RNA recruiting motif, thereby the guide nucleic acid guides the second DNA endonuclease to the target nucleic acid. An RNA recruiting motif may be linked to the 5′ end or to the 3′ end of the guide nucleic acid (e.g., a recruiting crRNA, a recruiting crDNA).

In some embodiments, a target nucleic acid contacted with the second complex may be further contacted with a DNA-dependent DNA polymerase (e.g., a second DNA-dependent DNA polymerase). In some embodiments, the DNA-dependent DNA polymerase may be comprised in the second complex.

The methods of the invention may further comprise contacting the target nucleic acid with a third complex, the third complex comprising a third sequence-specific DNA binding protein that is cable of binding to a third site on the target nucleic acid that is on a different strand from the first site and the second site, wherein the third sequence-specific DNA binding protein comprises nuclease or nickase activity, thereby improving the repair efficiency of the modifying of the target nucleic acid.

In some embodiments, the present invention provides a system for modifying a target nucleic acid comprising the first complex of the invention, a polynucleotide encoding the same, and/or the expression cassette or vector comprising the polynucleotide, wherein (a) the first sequence-specific DNA binding protein comprising DNA endonuclease activity binds to a first site on the target nucleic acid; (b) the first DNA-dependent DNA polymerase is capable of interacting with the first sequence-specific DNA binding protein and is recruited to the first sequence specific DNA binding protein and to the first site on the target nucleic acid, and (c) (i) the first DNA encoded repair template is linked to a first guide nucleic acid that comprises a spacer sequence having substantial complementarity to the first site on the target nucleic acid, thereby guiding the first DNA encoded repair template to the first site on the target nucleic acid, or (c)(ii) the first DNA encoded repair template is capable of interacting with the first sequence-specific DNA binding protein or the first DNA-dependent DNA polymerase and is recruited to the first sequence-specific DNA binding protein or the first DNA-dependent DNA polymerase and to the first site on the target nucleic acid, thereby modifying the target nucleic acid.

In some embodiments, a system for modifying a target nucleic acid is provided, the system comprising the first complex of the invention, a polynucleotide encoding the same, and/or the expression cassette or vector comprising the polynucleotide, wherein (a) the first sequence-specific DNA binding protein binds to a first site on the target nucleic acid, (b) the first DNA endonuclease is capable of interacting with the first sequence specific DNA binding protein and/or a guide nucleic acid and is recruited to the first sequence specific DNA binding protein and to the first site on the target nucleic acid; (c) the first DNA-dependent DNA polymerase is capable of interacting with the first sequence specific DNA binding protein and/or a guide nucleic acid and is recruited to the first sequence specific DNA binding protein and to the first site on the target nucleic acid; and (d) (i) the first DNA encoded repair template is linked to a guide nucleic acid that comprises a spacer sequence having substantial complementarity to the first site on the target nucleic acid, thereby guiding the first DNA encoded repair template to the first site on the target nucleic acid, or (d)(ii) the first DNA encoded repair template is capable of interacting with the first sequence-specific DNA binding protein or the first DNA-dependent DNA polymerase and is recruited to the sequence-specific DNA binding protein or the first DNA-dependent DNA polymerase and to the first site on the target nucleic acid, thereby modifying the target nucleic acid.

In some embodiments, the system of the invention for modifying a target nucleic acid may further comprise the second complex of the invention, a polynucleotide encoding the same, and/or an expression cassette and/or vector comprising the polynucleotide, wherein the second sequence-specific DNA binding domain binds to a second site proximal to the first site on the target nucleic acid and the second DNA-encoded repair template is recruited to the second sequence-specific DNA binding protein (via covalent or non-covalent interactions), thereby modifying the target nucleic acid.

A DNA-dependent DNA polymerase useful with this invention (e.g., a first and/or a second DNA-dependent DNA polymerase) may be any DNA dependent DNA polymerase. DNA-dependent DNA polymerases are well known in the art, a non-limiting list of which may be found at the Polbase website (polbase.neb.com). In some embodiments, a DNA-dependent DNA polymerase useful with this invention may comprise 3′-5′ exonuclease activity, 5′-3′ exonuclease activity and/or 5′-3′ RNA-dependent DNA polymerase activity. In some embodiments, a DNA-dependent DNA polymerase may be modified or engineered to remove one or more of 3′-5′ exonuclease activity, 5′-3′ exonuclease activity and 5′-3′ RNA-dependent DNA polymerase activity.

In some embodiments, a DNA-dependent DNA polymerase (e.g., a first and/or a second DNA-dependent DNA polymerase) with improve delivery and/or activity may be provided, the DNA-dependent DNA polymerase comprising a Klenow fragment or sub-fragment thereof. As an example, the E. coli Klenow fragment may be used, which is about 68 kDa in size or 62% the molecular weight of full length (109 kDa) DNA polymerase I.

A DNA-dependent DNA polymerase may be improved for temperature-sensitivity, processivity, and template affinity via fusion to a DNA binding domain. Thus, for example, a DNA-dependent DNA polymerase (e.g., a first and/or the second DNA-dependent DNA polymerase) may be fused to a sequence non-specific DNA binding protein to provide a DNA-dependent DNA polymerase having improved temperature-sensitivity, processivity, and/or template affinity. In some embodiments, a sequence non-specific DNA binding protein may be a sequence-nonspecific dsDNA binding protein that may include, but is not limited to, Sso7d from Sulfolobus solfataricus.

A DNA-dependent DNA polymerase (e.g., a first DNA-dependent DNA polymerase and/or the second DNA-dependent DNA polymerase) may be from a human, a yeast, a bacterium, or a plant. In some embodiments, a DNA-dependent DNA polymerase useful with the invention can include but is not limited to a DNA polymerase ε (e.g., human and yeast), DNA polymerase δ, E. coli polymerase I, Phusion® DNA polymerase, Vent® DNA polymerase, Vent (exo-)® DNA polymerase, Deep Vent® DNA polymerase, Deep Vent (exo-)® DNA polymerase, 9° Nm™ DNA polymerase, Q5® DNA polymerase, Q5U® DNA polymerase, Pfu DNA polymerase, and/or Phire™ DNA polymerase. In some embodiments, a DNA-dependent DNA polymerase may be a human DNA-dependent DNA polymerase ε, plant DNA-dependent DNA polymerase ε and/or yeast DNA-dependent DNA polymerase ε (see, e.g., SEQ ID NOS:48-58).

In some embodiments, a DNA-dependent DNA polymerase useful with this invention may exhibit high fidelity and/or high processivity. Processivity relates to the number of nucleotides incorporated in a single binding event of the polymerase to the template. In some cases, DNA-dependent DNA polymerases can have a processivity of more than 100 kb (e.g., about 100, 110, 120, 130, 140, 150, 160, 170, 180, 190, 200 kb or more, and any range or value therein). In some embodiments, a DNA-dependent DNA polymerase may exhibit a high distributive profile. Thus, a DNA-dependent DNA polymerase may be a high-fidelity DNA-dependent DNA polymerase and/or a high processivity DNA-dependent DNA polymerase. In some embodiments, a DNA-dependent DNA polymerase may be a distributive polymerase (e.g., a low processivity polymerase) or may be a DNA-dependent DNA polymerase having a high distributive profile.

A DNA-dependent DNA polymerase useful with the invention (e.g., the first DNA-dependent DNA polymerase and/or the second DNA-dependent DNA polymerase) may be the engineered DNA-dependent DNA polymerase of the present invention.

In some embodiments, a sequence-specific DNA binding protein (e.g., a first sequence-specific DNA binding protein, second sequence-specific DNA binding protein and/or third sequence-specific DNA binding protein) may be from a polynucleotide-guided endonuclease, a CRISPR-Cas effector protein, a protein-guided endonuclease (e.g., a zinc finger nuclease), a transcription activator-like effector nuclease (TALEN) and/or an Argonaute protein. In some embodiments, a sequence-specific DNA binding protein may comprise endonuclease or nickase activity and may be a polynucleotide-guided endonuclease, a CRISPR-Cas effector protein, a protein-guided endonuclease (e.g., a zinc finger nuclease), and/or a transcription activator-like effector nuclease (TALEN).

A DNA endonuclease (e.g., a first DNA endonuclease, second DNA endonuclease, and/or third DNA endonuclease) may be a nuclease and/or a nickase (capable of generating a double strand break or a single strand break in a nucleic acid, respectively). In some embodiments, a DNA endonuclease (e.g., a first DNA endonuclease, second DNA endonuclease, and/or third DNA endonuclease) may be an endonuclease (e.g., Fok1, or other similar endonuclease domain), a polynucleotide-guided endonuclease, a CRISPR-Cas effector protein, a protein-guided endonuclease (e.g., a zinc finger nuclease), and/or a transcription activator-like effector nuclease (TALEN).

In some embodiments, a sequence-specific DNA binding domain (e.g., a first sequence-specific DNA binding protein, second sequence-specific DNA binding protein and/or third sequence-specific DNA binding protein) and/or DNA endonuclease (e.g., a first DNA endonuclease, second DNA endonuclease and/or third DNA endonuclease) may be a CRISPR-Cas effector protein, optionally wherein the CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

Nonlimiting examples of a CRISPR-Cas effector protein can include a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site may have impaired activity or reduced activity as compared to the same CRISPR-Cas effector protein without the mutation. In some embodiments, a mutation in the nuclease active cite results in a CRISPR-Cas effector protein having nickase activity (e.g., Cas9n)

A CRISPR Cas9 effector protein or CRISPR Cas9 effector domain useful with this invention may be any known or later identified Cas9 polypeptide. In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. (See, e.g., SEQ ID NOs: 59-62).

Cas12a is a Type V Clustered Regularly Interspaced Short Palindromic Repeats (CRISPR)-Cas nuclease. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity.

In some embodiments, a peptide tag (e.g., an epitope, a peptide repeat unit) useful with this invention for recruiting polypeptides to selected locations (e.g., target nucleic acid, site on a target nucleic acid) may comprise 1 or 2 or more copies of a peptide tag (epitope, multimerized epitope) (e.g., 1, 2, 3, 4, 5, 6, 7, 8, 9, 10, 11, 12, 13, 14, 15, 16, 17, 18, 19, 20, 21, 22, 23, 24, 25 or more copies (repeat units). In some embodiments, a peptide tag useful with this invention can include, but is not limited to, a GCN4 peptide tag (e.g., Sun-Tag) (see, e.g., SEQ ID NOs:23-24), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. In some embodiments, the peptide tag may be a GCN4 peptide tag. In some embodiments, a peptide tag may comprise two or more copies of the peptide tag (a peptide repeat; e.g., two or more tandem copies; e.g., tandem copies of GCN4).

In some embodiments, an affinity polypeptide capable of binding a peptide tag can include, but is not limited to, an antibody, optionally a scFv antibody that is capable of binding a peptide tag (e.g., a GCN4 peptide tag (see, e.g., SEQ ID NO:25), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope), an affibody, an anticalin, a monobody, and/or a DARPin, each of which are capable of binding a peptide tag (e.g., a GCN4 peptide tag, a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope).

In some embodiments of the invention, a guide nucleic acid (CRISPR nucleic acid, crRNA, crDNA) may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs, whereby the guide nucleic acid linked to one or more RNA recruiting motifs may be used to recruit one or more polypeptides that are fused to an affinity polypeptide that is capable of interacting with/binding an RNA recruiting motif linked to the guide.

In some embodiments, an RNA recruiting motif and affinity polypeptide capable of interacting with the RNA recruiting motif (e.g., a corresponding affinity polypeptide) may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein and/or a synthetic RNA-aptamer and the aptamer ligand as the corresponding affinity polypeptide (see, e.g., SEQ ID NOs:26-36). In some embodiments, an RNA recruiting motif and its corresponding affinity polypeptide useful with the invention may be an MS2 phage operator stem-loop and the affinity polypeptide MS2 Coat Protein (MCP), and/or a PUF binding site (PBS) and the affinity polypeptide Pumilio/fem-3 mRNA binding factor (PUF).

As described herein, polypeptides of the invention may be fusion proteins comprising one or more polypeptides linked to one another. In some embodiments, the fusion is via a linker. In some embodiments, a linker may be an amino acid or peptide linker. In some embodiments, a peptide linker may be about 2 to about 100 amino acids (residues) in length. In some embodiments, a peptide linker may be a GS linker.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer. In some embodiments, as described herein, a guide RNA may include a template for editing and a primer binding site. In some embodiments, a guide RNA may include a region or sequence on its 5′ end or 3′ end that is complementary to an editing template (a reverse transcriptase template), thereby recruiting the editing template to the target nucleic acid.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas nuclease encoded by the nucleic acid constructs of the invention that encode a base editor. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue): W52-7). In some embodiments, a repeat sequence or portion thereof is linked at its 3′ end to the 5′ end of a spacer sequence, thereby forming a repeat-spacer sequence (e.g., guide RNA, crRNA).

In some embodiments, a repeat sequence comprises, consists essentially of, or consists of at least 10 nucleotides depending on the particular repeat and whether the guide RNA comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 23 nucleotides in length.

In some embodiments, the 5′ region of a spacer sequence of a guide RNA may be identical to a target DNA, while the 3′ region of the spacer may be substantially complementary to the target DNA (e.g., Type V CRISPR-Cas), or the 3′ region of a spacer sequence of a guide RNA may be identical to a target DNA, while the 5′ region of the spacer may be substantially complementary to the target DNA (e.g., Type II CRISPR-Cas), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of an organism's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide RNA of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome, an animal genome, a bacterial genome, a fungal genome, and the like). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide RNAs, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
    5′-NNNNNNNNNNNNNNNNNNN-3′ RNA Spacer (SEQ ID NO: 37)
          |||||||||||||||||||
3′AAANNNNNNNNNNNNNNNNNNN-5′ Target strand (SEQ ID NO: 38)
    ||||
5′TTTNNNNNNNNNNNNNNNNNNN-3′ Non-target strand (SEQ ID NO: 39
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST® searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors encoding the same but which have not been codon optimized for expression in a plant.

In some embodiments, the invention provides cells comprising one or more polynucleotides, guide nucleic acids, nucleic acid constructs, expression cassettes or vectors of the invention.

When used in combination with guide nucleic acids, the nucleic acid constructs of the invention of the invention may be used to modify a target nucleic acid. A target nucleic acid may be contacted with a nucleic acid construct of the invention prior to, concurrently with or after contacting the target nucleic acid with the guide nucleic acid. In some embodiments, the nucleic acid constructs of the invention and a guide nucleic acid may be comprised in the same expression cassette or vector and therefore, a target nucleic acid may be contacted concurrently with the nucleic acid constructs of the invention and guide nucleic acid. In some embodiments, the nucleic acid constructs of the invention and a guide nucleic acid may be in different expression cassettes or vectors and thus, a target nucleic acid may be contacted with the nucleic acid constructs of the invention prior to, concurrently with, or after contact with a guide nucleic acid.

A target nucleic acid of any organism or cell thereof may be modified (e.g., mutated, e.g., base edited, cleaved, nicked, etc.) using the nucleic acid constructs of the invention (e.g., the polypeptides and complexes (e.g., sequence specific DNA binding proteins, DNA-dependent DNA polymerases (e.g., engineered DNA-dependent DNA polymerases), DNA endonucleases, DNA encoded repair templates, guide nucleic acids, and the like) and polynucleotides, expression cassettes, and/or vectors encoding the same).

In some embodiments, a target nucleic acid of any plant or plant part may be modified (e.g., mutated, e.g., base edited, cleaved, nicked, etc.) using the nucleic acid constructs of the invention (e.g., the polypeptides and complexes (e.g., sequence specific DNA binding proteins, DNA-dependent DNA polymerases (e.g., engineered DNA-dependent DNA polymerases), DNA endonucleases, DNA encoded repair templates, guide nucleic acids, and the like) and polynucleotides, expression cassettes, and/or vectors encoding the same). Any plant (or groupings of plants, for example, into a genus or higher order classification) may be modified using the nucleic acid constructs of this invention including an angiosperm, a gymnosperm, a monocot, a dicot, a C3, C4, CAM plant, a bryophyte, a fern and/or fern ally, a microalgae, and/or a macroalgae. A plant and/or plant part useful with this invention may be a plant and/or plant part of any plant species/variety/cultivar. The term “plant part,” as used herein, includes but is not limited to, embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell can be in the form of an isolated single cell or can be a cultured cell or can be a part of a higher-organized unit such as, for example, a plant tissue or a plant organ.

Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, sunflower, raspberry, blackberry, black raspberry and/or cherry. In some embodiments, the nucleic acid constructs of the invention and/or expression cassettes and/or vectors encoding the same may be used to modify a Rubus spp. (e.g., blackberry, black raspberry, boysenberry, loganberry, raspberry, e.g., caneberry), a Vaccinium spp. (e.g., cranberry), a Ribes spp. (e.g., gooseberry, currants (e.g., red currant, black currant)), or a Fragaria spp. (e.g., strawberry).

The present invention further comprises a kit or kits to carry out the methods of this invention. A kit of this invention can comprise reagents, buffers, and apparatus for mixing, measuring, sorting, labeling, etc, as well as instructions and the like as would be appropriate for modifying a target nucleic acid.

In some embodiments, the invention provides a kit comprising one or more nucleic acid constructs of the invention and/or expression cassettes and/or vectors comprising the same (e.g., comprising or encoding the polypeptides/complexes of the invention), with optional instructions for the use thereof. In some embodiments, a kit may further comprise a CRISPR-Cas guide nucleic acid (corresponding to the CRISPR-Cas nuclease encoded by the polynucleotide of the invention) and/or expression cassette and/or vector comprising the same. In some embodiments, the guide nucleic acid may be provided on the same expression cassette and/or vector as a nucleic acid construct of the invention. In some embodiments, the guide nucleic acid may be provided on a separate expression cassette or vector from that comprising the nucleic acid construct of the invention.

In some embodiments, the kit may further comprise a nucleic acid construct encoding a guide nucleic acid, wherein the construct comprises a cloning site for cloning of a nucleic acid sequence identical or complementary to a target nucleic acid sequence into backbone of the guide nucleic acid.

In some embodiments, a nucleic acid construct of the invention and/or an expression cassette and/or vector comprising the same, may further encode one or more selectable markers useful for identifying transformants (e.g., a nucleic acid encoding an antibiotic resistance gene, herbicide resistance gene, and the like).

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1. In Vivo Precision Templated Editing

Precision templated editing via fusion of DNA-dependent DNA polymerase to CRISPR protein in human cells can be demonstrated by co-transfecting a mix of components into the human cell line HEK293T. The mix of components includes a recipient plasmid, which contains a copy of mutant EGFP gene driven by CMV promoter; a single stranded DNA repair template containing the correcting sequence for the mutant EGFP flanked by 100-200 nt of homologous sequence to facilitate binding of template to the target site; a second plasmid that expresses fusion protein of a CRISPR protein (e.g., eCas9, nCas9 (D10A), or nCas9 (H840A)) and a DNA-dependent DNA polymerase of interest (e.g., Pol I from E.coli), where the fusion of DNA-dependent DNA polymerase to the N-or C-terminus CRISPR protein is via a linker; a third plasmid that expresses a guide RNA that targets the mutant EGFP sequence. As a control, the second plasmid will be replaced with a plasmid that expresses only the relative CRISPR protein. Desired templated editing events will be identified with flow cytometry, as the mutant EGFP is corrected to a functional copy of EGFP resulting in a green fluorescent phenotype.

Alternatively, the DNA repair template and the third plasmid that expresses guide RNA (or guide DNA) can be replaced by a plasmid that expresses a retron reverse transcriptase and chimeric guide RNA (or chimeric guide DNA) with retron scaffold containing the repair template.

### Example 2. Precision Templated Editing Via CRISPR Protein and DNA-Dependent DNA Polymerase In Vitro

Precision templated editing via CRISPR protein and DNA-dependent DNA polymerase in vitro. Commercially available DNA-dependent DNA polymerases are evaluated in vitro for their potential to conduct templated replacement of target DNA sequence from a nick introduced by CRISPR nickase nCas9 (H840A). Non-limiting examples of DNA-dependent DNA polymerases for evaluation include Q5 High-Fidelity DNA polymerase, Phusion® High-Fidelity DNA polymerase, Hemo Klen Taq DNA polymerase, Bst 2.0 DNA polymerase, Bsu DNA polymerase, Phi29 DNA polymerase, T7 DNA polymerase, Therminator™ DNA polymerase, Klenow Fragment (3′->5′ exo-), Vent (exo-).

A 2 kb DNA fragment that contains a Cas9 binding site in the center of the fragment is used as the recipient, a single stranded DNA repair template of ˜100nt is used to introduce mismatches to the recipient adjacent to the Cas9 target site. A mixture of recipient DNA, repair template, nCas9 (H840A) protein and guide RNA, and a DNA-dependent DNA polymerase is incubated at 37° C. or 25° C. Desired repair products containing mismatches can be digested by T7 endonuclease I, separated from other products and quantitated by gel electrophoresis.

### Example 3. Precision Templated Editing Via MS2 RNA Loop Recruitment of DNA-Dependent DNA Polymerase to Target Site

Precision templated editing via MS2 RNA loop recruitment of DNA-dependent DNA polymerase to target site can be demonstrated by co-transfecting a mix of components into the human cell line HEK293T. The mix of components includes a recipient plasmid that contains a copy of mutant EGFP gene driven by CMV promoter; a single stranded DNA repair template containing the correcting sequence for the mutant EGFP flanked by 100-200 nt of homologous sequence to facilitate binding of template to the target site; a second plasmid that expresses a DNA-dependent DNA polymerase of interest (e.g., Pol I from E.coli) with a MCP domain fused to its N-terminus via a linker; a third plasmid that expresses a guide RNA that targets the mutant EGFP sequence, where the guide nucleic acid scaffold is modified to contain MS2 stem loop that interacts with the MCP domain; and a fourth plasmid that expresses the CRISPR protein (e.g., eCas9, nCas9 (D10A), or nCas9 (H840A)). As a control, the second plasmid is omitted from the transfection mix. Desired templated editing events are identified with flow cytometry, as the mutant EGFP is corrected to a functional copy of EGFP. Alternatively, the DNA repair template and the third plasmid that expresses MS2 guide RNA can be replaced by a plasmid that expresses a retron reverse transcriptase and chimeric MS2 guide RNA with retron scaffold containing the repair template.

### Example 4. Precision Templated Editing Via PUF-Binding Site (PBS) RNA Aptamer Recruitment of DNA-Dependent DNA Polymerase to Target Site

Precision templated editing via PUF-binding site (PBS) RNA aptamer recruitment of DNA-dependent DNA polymerase to target site can be demonstrated by co-transfecting a mix of components into the human cell line HEK293T. The mix of components includes a recipient plasmid that contains a copy of mutant EGFP gene driven by CMV promoter; a single stranded DNA repair template containing the correcting sequence for the mutant EGFP flanked by 100-200 nt of homologous sequence to facilitate binding of template to the target site; a second plasmid that expresses a DNA-dependent DNA polymerase of interest (eg. Pol I from E.coli) with a PUF domain fused to its N-terminus via a linker; a third plasmid that expresses a guide RNA that targets the mutant EGFP sequence, where the guide RNA scaffold is modified to contain PUF-binding site that interacts with the PUF domain; and a fourth plasmid that expresses the CRISPR protein (eg. eCas9, nCas9 (D10A), or nCas9 (H840A)). As a control, the second plasmid is omitted from the transfection mix. Desired templated editing events are identified with flow cytometry, as the mutant EGFP is corrected to a functional copy of EGFP. Alternatively, the DNA repair template and the third plasmid that expresses guide RNA with PBS can be replaced by a plasmid that expresses a retron reverse transcriptase and chimeric guide RNA with PBS and retron scaffold containing the repair template.

### Example 5. Precision Templated Editing Via PUF-Binding Site (PBS) RNA Aptamer Recruitment of DNA-Dependent DNA Polymerase to Target Site

Precision templated editing via antibody/epitope recruitment of DNA-dependent DNA polymerase to target site can be demonstrated by co-transfecting a mix of components into the human cell line HEK293T. The mix of components includes: a recipient plasmid that contains a copy of mutant EGFP gene driven by CMV promoter; a single stranded DNA repair template containing the correcting sequence for the mutant EGFP flanked by 100-200 nt of homologous sequence to facilitate binding of template to the target site; a second plasmid that expresses a DNA-dependent DNA polymerase of interest (e.g., Pol I from E.coli) with a scFV domain fused to its N-terminus via a linker; a third plasmid that expresses a guide RNA that targets the mutant EGFP sequence; and a fourth plasmid that expresses the CRISPR protein (eg. eCas9, nCas9 (D10A), or nCas9 (H840A)), with 8 copies of GCN4 tags fused to its C-terminus. As a control, the second plasmid is omitted from the transfection mix. Desired templated editing events are identified with flow cytometry, as the mutant EGFP is corrected to a functional copy of EGFP. Alternatively, the DNA repair template and the third plasmid that expresses guide RNA can be replaced by a plasmid that expresses a retron reverse transcriptase and chimeric guide RNA with retron scaffold containing the repair template.

### Example 6. Precision Templated Editing Via PUF-Binding Site (PBS) RNA Aptamer Recruitment of DNA-Dependent DNA Polymerase to Target Site

Precision templated editing and site directed integration of long fragment via recruitment of DNA-dependent DNA polymerase in plants can be demonstrated by inserting an EGFP gene (˜700 bp) in frame into an exon of a highly expressed gene (e.g., actin). In this experiment design, two T-DNAs will be co transformed into plant tissue. The first T-DNA contains a tool cassette that expressed CRISPR protein and DNA-dependent DNA polymerase in the correct architecture for efficient recruitment of the DNA-dependent DNA polymerase to target site, and a guide cassette that expresses guide RNA targeting the last exon of actin in the necessary configuration for protein recruitment. The second T-DNA contains repair template that encodes full length of EGFP and in-frame deletion of the stop codon in the targeted exon. This repair template is flanked by target sites recognized by the guide RNA expressed in the first T-DNA. Desired site directed integration of the EGFP results in expression of EGFP driving by the promoter of actin gene, while random integration does not yield EGFP express due to lack of promoter. Frequency of site directed integration can be quantitated by microscopy. Alternatively, the first T-DNA will only express the tool cassette, the second T-DNA will contain a retron reverse transcriptase cassette and a chimeric guide RNA cassette that encodes repair template in a retron scaffold attached to the guide RNA scaffold.

### Example 7. Recruitment and Optimization of DNA-Dependent DNA Polymerase

As described in the above examples and more generally herein, many different methods may be used to recruit a DNA-dependent DNA polymerase to an editing site. For example, DNA-dependent DNA polymerase can be fused to the C- or N-terminus of CRISPR protein via a flexible linker, such as in the architecture of base editors. Alternatively, DNA-dependent DNA polymerase can be recruited to the target broken or nicked DNA via interaction with guide RNA (eg. MS2 loop) or CRISPR protein (eg. SunTag).

The function of a DNA-dependent DNA polymerase may be improved/optimized in any number of ways including, but not limited to, by removing 3′-5′ exonuclease, 5′-3′ exonuclease and/or 5′-3′ RNA-dependent DNA polymerase activities. A DNA dependent DNA polymerase may further comprise the Klenow fragment or other sub-fragment of the protein. Klenow fragments or other active fragments may be useful for delivery or activity purposes. As an example, the E. coli Klenow fragment is 68 kDa or 62% the molecular weight of the full (109 kDa) DNA polymerase I.

Protein domain fusions to the DNA-dependent DNA polymerase enzyme can have significant effects on the temperature-sensitivity and processivity of the editing system. The DNA-dependent DNA polymerase enzyme can be improved for temperature-sensitivity, processivity, and template affinity through fusions to DNA binding domains (DBDs). These DBDs may have sequence specificity, non-specificity or sequence preferences. A range of affinity distributions may be beneficial to editing in different cellular and in vitro environments. Adding one or more DBD to the DNA-dependent DNA polymerase enzyme can result in increased affinity, increased or decreased sequence specificity, and/or promote cooperativity. One particular DBD known to increase processivity of DNA-dependent DNA polymerases is sequence-nonspecific dsDNA binding protein Sso7d, from Sulfolobus solfataricus (Wang, 2004). The dsDNA binding protein may be fused to either the C-terminus, N-terminus or flexible loop of the polymerase. Increased processivity can be demonstrated by inserting a larger reporter gene such as tdTomato (˜1500bp) in frame into an exon of a highly expressed gene (eg. actin). For example, two T-DNAs may be co transformed into plant tissue. The first T-DNA contains a tool cassette that expressed CRISPR protein and DNA-dependent DNA polymerase::ssDBD in the correct architecture for efficient recruitment of the DNA-dependent DNA polymerase::ssDBD to target site, and a guide cassette that expresses guide RNA targeting the last exon of actin in the necessary configuration for protein recruitment. The second T-DNA contains repair template that encodes full length of tdTomato (or other reporter) and in-frame deletion of the stop codon in the targeted exon. This repair template is flanked by target sites recognized by the guide RNA expressed in the first T-DNA. Desired site directed integration of the tdTomato (or other reporter) results in expression of tdTomato driving by the promoter of actin gene, while random integration does not yield tdTomato express due to lack of promoter. Frequency of site directed integration can be quantitated by microscopy.

### Example 8. CRISPR Polypeptides

This invention takes advantage of high processivity DNA-dependent DNA polymerase to rapidly initiate DNA synthesis primed by the 3′ end of broken or nicked target DNA annealed to a provided repair template. Cas9 nuclease and nickase, and Cas12a nuclease and nickase and other CRISPR-Cas effector polypeptides can be used to produce 3′DNA target ends. Successful incorporation of repair templates, particularly templates having a large size, can depend on the ability of DNA-dependent DNA polymerase to move along the DNA templates away from the broken or nick site. It is possible that fusion directly to Cas9 protein, which may remain bound to cleaved or nicked DNA, may hinder the movement of DNA-dependent DNA polymerase. For that reason, a Cas9 with reduced binding affinity to DNA such as eCas9 (three amino acid mutations (K848A, K1003A, R1060A)⁴) nuclease or nickase may be used. Alternatively, non-covalent recruitment of the polymerase to the CRISPR complex may be used to maximize the opportunity for the polymerase to function without steric inhibition or mobility constraints. Several covalent and non-covalent recruitment strategies are described herein. For example, Cpf1/Cas12a has a longer seed sequence for stable binding (17-bp vs. 9-10-bp for Cas9) this indicates a lower affinity for target DNA (Jeon et. al, 2018), consistent with the lower off-target rate of editing found with Cpf1. The lower affinity, of Cpf1 relative to Cas9, for target DNA may be an advantage for polymerase fusions requiring mobility of the editing tool.

### Example 9. Repair Template Recruitment

In human cell experiments, a repair template may be recruited through a number of different strategies, including, but not limited to: 1) interaction between PCV domain that is fused to CRISPR protein, and the PCV recognition sites embedded in the repair template; and 2) msDNA encoding repair template produced from chimeric retron-guide RNA scaffold and tethered to the guide RNA scaffold.

### Example 10. Genome Editing in Plants

In editing of plants, various methods of repair template delivery can be used and these can vary depending on transformation method. For example, for Agrobacterium-mediated plant transformation, VirD2 or VirE2 mediated T-DNA recruitment may be used or msDNA, and for particle bombardment, a HUH tagging system and msDNA may be used.

### Example 11. Editing in Human Cells

Eukaryotic HEK293T (ATCC CRL-3216) cells were cultured in Dulbecco's Modified Eagle's Medium plus GlutaMax (ThermoFisher) supplemented with 10% (v/v) FBS (FBS), at 37° C. with 5% CO2. HEK293T cells were seeded on 48-well collagen-coated BioCoat plates (Corning). Cells were transfected at about 70% confluency. DNA was transfected using 1.5 μl of Lipofectamine 3000 (ThermoFisher Scientific) per well according to the manufacturer's protocol. RNP was transfected using 1.5 μl of RNAiMAX (ThermoFisher Scientific) per well according to the manufacturer's protocol. Genomic DNA from transfected cells were obtained after 3 days and precise editing was detected and quantified using high-throughput Illumina amplicon sequencing.

To test DNA polymerase-mediated elongation of DNA template, the following was done. HEK293T cells were first transfected with 1 μg of DNA encoding various DNA-dependent DNA polymerases including Klentaq, Therminator, Pfu-Ssod7, Klenow, E. coli poll, HU pol E (N-term), yeast pol E under constitutive CMV promoter (see, e.g., SEQ ID NOs:48-58, 88-94). All DNA-dependent DNA polymerases were augmented with at least one SV40 nuclear localization sequence to ensure importation into the nucleus. After 4 h, the cells were placed under a fresh media. Then Cas12a RNP complexes (see, e.g., SEQ ID NO:75) containing various synthetic crRNA extensions (see, e.g., SEQ ID NOs:78, 79, 82, 83, 86, 87) were transfected into the cells. DNA extension encoding a homology arm downstream of Cas12a cut site and a template sequence encoding a desired edit was conjugated to the crRNA via chemical synthesis (Integrated DNA Technologies). Two different homology lengths were tested (PBS; 24 bp and 36 bp) and the length of the template containing the desired edit (RTT) was 36 base pairs (Table 2). Three different spacers were used to test the system (PWsp137 (SEQ ID NO:76), PWsp453 (SEQ ID NO:80), PWsp454 (SEQ ID NO:84) (Table 2). For all the constructs, the template contained precise dinucleotide changes at position −2 and −3 of the spacer into adenines (TT to AA), with the PAM sequence (TTTV) corresponding to position −4, −3, −2, and −1.

PWsp137 Target Sequence:
SEQ ID NO: 76
CCUCACUCCUGCUCGGUGAAUUU
PWsp137 crRNA-No extension:
SEQ ID NO: 77
AAUUUCUACUAAGUGUAGAUCCUCACUCCUGCUCGGUGAAUUU
PWsp137 crRNA-PBS 24 bp; RTT 36 bp:
SEQ ID NO: 78
AAUUUCUACUAAGUGUAGAUCCUCACUCCUGCUCGGUGAAUUU
CTGGGGCCGTAACCCTCACTCCTGCTCGGTGAATTTGGCTCAGCAGGCACCTGCCTCAGC
PWsp137 crRNA-PBS 36 bp; RTT 36 bp:
SEQ ID NO: 79
AAUUUCUACUAAGUGUAGAUCCUCACUCCUGCUCGGUGAAUUU
CTGGGGCCGTAACCCTCACTCCTGCTCGGTGAATTTGGCTCAGCAGGCACCTGCCTCAGCTGCT
CACTTGAG
PWsp453 Target Sequence:
SEQ ID NO: 80
UAUGAGUUACAACGAACACCUCA
PWsp453 crRNA-No extension:
SEQ ID NO: 81
AAUUUCUACUAAGUGUAGAUUAUGAGUUACAACGAACACCUCA
PWsp453 crRNA-PBS 24 bp; RTT 36 bp:
SEQ ID NO: 82
AAUUUCUACUAAGUGUAGAUUAUGAGUUACAACGAACACCUCA
GGAACTCAGTAAATATGAGTTACAACGAACACCTCAGGTAATGACTAAGATGACTGCCAA
PWsp453 crRNA-PBS 36 bp; RTT 36 bp:
SEQ ID NO: 83
AAUUUCUACUAAGUGUAGAUUAUGAGUUACAACGAACACCUCA
GGAACTCAGTAAATATGAGTTACAACGAACACCTCAGGTAATGACTAAGATGACTGCCAAGGGG
CATATGAG
PWsp454 Target Sequence:
SEQ ID NO: 84
CACGUCUCAUAUGCCCCUUGGCA
PWsp454 crRNA-No extension:
SEQ ID NO: 85
AAUUUCUACUAAGUGUAGAUCACGUCUCAUAUGCCCCUUGGCA
PWsp454 crRNA-PBS 24 bp; RTT 36 bp:
SEQ ID NO: 86
AAUUUCUACUAAGUGUAGAUCACGUCUCAUAUGCCCCUUGGCA
GTATCCCAGTAAACACGTCTCATATGCCCCTTGGCAGTCATCTTAGTCATTACCTGAGGT
PWsp454 crRNA-PBS 36 bp; RTT 36 bp:
SEQ ID NO: 87
AAUUUCUACUAAGUGUAGAUCACGUCUCAUAUGCCCCUUGGCA
GTATCCCAGTAAACACGTCTCATATGCCCCTTGGCAGTCATCTTAGTCATTACCTGAGGTGTTCG
TTGTAAC

We detected precise editing without any side products using DNA polymerases in conjunction with Cas12a RNP that contains DNA extensions on crRNA (Table 2). Precise editing was detected in all 3 spacers tested (Table 2). As indel rates are expected to be efficient from a LbCas12a RNPs (5-50% editing efficiency in 293T-see, e.g., Liu et al. Nucleic Acids Res. 47(8):4169-4180 (2019)), our low (˜1%) indel rates (Table 3) suggest that the two rounds of transfection in our experiment significantly decreased efficiency of the delivery system.

Given that precise editing rates in our experiment were similar to the indel rates (Table 3 and Table 4) suggests that precise editing via DNA-dependent DNA polymerase is potentially quite efficient for precise editing. When background levels of precise editing are subtracted and precise edits are normalized to the rate of indel edits (to normalize for transfection and viability rates), it is apparent that addition of the DNA polymerases and template lead to substantial increases in precise edits relative to the No DNA polymerase control at most spacer sites and PBS lengths (Table 4).

TABLE 2
Precise editing detected in NGS amplicon sequencing from treated samples expressed as % of total reads.

| | | | | | % Precise Editing (TT to AA | | | | |
|---|---|---|---|---|---|---|---|---|---|
| | % indels | | | | at position −2 and −3) | | | | |
| | No Extension | | | PBS 24bp; RTT 36bp | | | PBS 36bp; RTT 36bp | | |
| Polymerase | PWsp | PWsp | PWsp | PWsp | PWsp | PWsp | PWsp | PWsp | PWsp |
| Used | 137 | 453 | 454 | 137 | 453 | 454 | 137 | 453 | 454 |
| No DNA | 0.247 | 0 | 0 | 0.191 | 0.105 | N/D* | 0.154 | 0.062 | 0 |
| Polymerase | | | | | | | | | |
| Klentaq | 0 | 0. | 0 | 0 | 0.058 | 0 | 0.172 | 0.049 | N/D |
| Therminator | 0 | N/D | 0 | 0 | 0 | 0.04 | 0 | 0.018 | 0 |
| Pfu-Ssod7 | 0 | 0 | 0 | 0.352 | N/D | 0 | No Data | 0.041 | 0 |
| Klenow | 0 | 0 | 0 | 0.364 | N/D | 0 | N/D | 0.07 | 0.057 |
| E. Coli poll | N/D | 0 | 0 | 0.074 | 0.012 | 0 | N/D | 0.038 | 0.047 |
| HU pol E (N-term) | 0 | N/D | 0 | 0.152 | 0 | 0.043 | N/D | 0.022 | N/D |
| yeast pol E | 0 | N/D | 0 | 0.258 | 0.029 | 0.083 | N/D | 0.076 | N/D |

*N/D is no data

TABLE 3
Percent indels per total reads NGS amplicon sequencing from treated samples. N/D is no data

| | % indels | | | | | | | | |
|---|---|---|---|---|---|---|---|---|---|
| | No Extension | | | PBS 24bp; RTT 36bp | | | PBS 36bp; RTT 36bp | | |
| Polymerase | PWsp | PWsp | PWsp | PWsp | PWsp | PWsp | PWsp | PWsp | PWsp |
| Used | 137 | 453 | 454 | 137 | 453 | 454 | 137 | 453 | 454 |
| No DNA | 0.64 | 0.48 | 0.34 | 0.01 | 0.25 | No | 0.31 | 0.04 | 0.11 |
| Polymerase | | | | | | Data | | | |
| Klentaq | 0.78 | 0.37 | 0.56 | 0.19 | 0.08 | 0.11 | 0.03 | 0.1 | N/D* |
| Therminator | 1.30 | N/D | 0.30 | 0.51 | 0. | 0 | 0.15 | 0.09 | 0 |
| Pfu-Ssod7 | 0.21 | 0.56 | 0 | 0.09 | N/D | 0 | N/D | 0.04 | 0.02 |
| Klenow | 1.32 | 0.46 | 0 | 0.55 | N/D | 0 | N/D | 0.21 | 0.11 |
| E. Coli poll | N/D | 0.49 | 0.28 | 0.25 | 0 | 0.15 | N/D | 0.1 | 0.12 |
| HU pol E (N- | 0.460 | N/D | 0 | 0.04 | 0 | 0.02 | N/D | 0.08 | N/D |
| term) | | | | | | | | | |
| yeast pol E | 1.100 | N/D | 0.12 | 0.11 | 0.05 | 0.08 | N/D | 0.10 | N/D |

*N/D is no data

TABLE 4
Precise edit reads normalized to the indel editing rate of each
sample after subtracting background rates of precise editing
without a template extension (expressed as a fold change
relative to the indel rate). N/D is no data

| | Normalized Precise Editing (TT to AA | | | | | |
|---|---|---|---|---|---|---|
| | at position −2 and −3) (precise/indels) | | | | | |
| | PBS 24bp; RTT 36bp | | | PBS 36bp; RTT 36bp | | |
| Polymerase | PWsp | PWsp | PWsp | PWsp | PWsp | PWsp |
| Used | 137 | 453 | 454 | 137 | 453 | 454 |
| No DNA | 0 | 0.42 | N/D* | 0 | 1.561 | 0 |
| Polymerase | | | | | | |
| Klentaq | 0 | 0.722 | 0 | 5.749 | 0.488 | N/D |
| Therminator | 0 | — | — | 0 | 0.2 | — |
| Pfu-Ssod7 | 3.916 | N/D | — | N/D | 1.025 | 0 |
| Klenow | 0.662 | N/D | — | N/D | 0.332 | 0.517 |
| E. Coli poll | 0.297 | — | 0 | N/D | 0.384 | 0.389 |
| HU pol E (N- | 3.8 | — | 2.142 | N/D | 0.276 | N/D |
| term) | | | | | | |
| yeast pol E | 2.348 | 0.577 | 1.04 | N/D | 0.764 | N/D |

*N/D is no data

The foregoing is illustrative of the present invention and is not to be construed as limiting thereof. The invention is defined by the following claims, with equivalents of the claims to be included therein.

That which is claimed is:

1. A method of modifying a target nucleic acid, the method comprising:
contacting the target nucleic acid with:
(a) a sequence-specific DNA binding protein that binds to a site on a target nucleic acid, wherein the sequence-specific DNA binding protein is from a CRISPR-Cas effector protein;
(b) a DNA endonuclease having nickase or nuclease activity that introduces a single strand nick or a double strand break in the target nucleic acid;
(c) an RNA: DNA hybrid guide comprising an RNA guide nucleic acid and a DNA encoded repair template, wherein the DNA encoded repair template is linked to the RNA guide nucleic acid, thereby guiding the DNA encoded repair template to the target nucleic acid; and
(d) a DNA-dependent DNA polymerase that initiates strand synthesis copying genetic information from the DNA encoded repair template to the target nucleic acid, wherein the DNA-dependent DNA polymerase is recruited to the sequence-specific DNA binding protein, thereby modifying the target nucleic acid.

2. The method of claim 1, wherein the sequence-specific DNA binding protein, the DNA-dependent DNA polymerase, the DNA endonuclease, and the DNA encoded repair template form a complex.

3. The method of claim 1, wherein the DNA endonuclease is from a CRISPR-Cas effector protein or is a Fok1 endonuclease.

4. The method of claim 1, wherein the RNA guide nucleic acid is linked to an RNA-recruiting motif and the DNA-dependent DNA polymerase is fused to an affinity polypeptide that binds to the RNA recruiting motif, thereby guiding the DNA-dependent DNA polymerase to the target nucleic acid.

5. The method of claim 4, wherein the RNA recruiting motif is linked to the 5′ end or to the 3′ end of the RNA guide nucleic acid.

6. The method of claim 4, wherein the RNA recruiting motif and a corresponding affinity polypeptide are a telomerase Ku binding motif and the affinity polypeptide of Ku; a telomerase Sm7 binding motif and the affinity polypeptide of Sm7; an MS2 phage operator stem-loop and the affinity polypeptide MS2 Coat Protein (MCP), a PP7 phage operator stem-loop and the affinity polypeptide PP7 Coat Protein (PCP); an SfMu phage Com stem-loop and the affinity polypeptide Com RNA binding protein; a PUF binding site (PBS) and the affinity polypeptide Pumilio/fem-3 mRNA binding factor (PUF); and/or a synthetic RNA-aptamer and the corresponding aptamer ligand.

7. The method of claim 4, wherein the RNA recruiting motif and corresponding affinity polypeptide are an MS2 phage operator stem-loop and the affinity polypeptide MS2 Coat Protein (MCP), and/or a PUF binding site (PBS) and the affinity polypeptide Pumilio/fem-3 mRNA binding factor (PUF).

8. The method of claim 1, wherein the CRISPR-Cas effector protein is from a Type II CRISPR-Cas system or a Type V CRISPR-Cas system.

9. The method of claim 1, wherein the CRISPR-Cas effector protein is a Cas9 effector protein or a Cas12 effector protein.

10. The method of claim 1, further comprising contacting the target nucleic acid with a complex comprising:
(a) a second sequence-specific DNA binding protein that binds to a second site on the target nucleic acid; and
(b) a DNA-encoded repair template.

11. The method of claim 10, wherein the target nucleic acid is further contacted with a second DNA endonuclease, wherein the second DNA endonuclease introduces a single stranded nick or a double strand break into the target nucleic acid or wherein the second sequence-specific DNA binding protein comprises DNA endonuclease activity that introduces a single stranded nick or a double strand break into the target nucleic acid.

12. The method of claim 10, further comprising contacting the target nucleic acid with a second DNA-dependent DNA polymerase that initiates strand synthesis.

13. The method of claim 10, further comprising contacting the target nucleic acid with a third complex, the third complex comprising a third sequence-specific DNA binding protein that binds to a third site on the target nucleic acid that is on a different strand from the site and the second site, wherein the third sequence-specific DNA binding protein comprises nuclease or nickase activity and introduces a single stranded nick into the third site on the target nucleic acid, thereby improving the repair efficiency of the modifying of the target nucleic acid.

14. The method of claim 1, wherein the DNA-dependent DNA polymerase is a high fidelity DNA polymerase.

15. The method of claim 1, wherein the DNA endonuclease is a CRISPR-Cas effector protein, optionally wherein the CRISPR-Cas effector protein is the CRISPR-Cas effector protein of (a) or is a different CRISPR-Cas effector protein.

16. The method of claim 1, wherein the sequence-specific DNA binding protein is fused to a peptide tag and the DNA-dependent DNA polymerase is fused to an affinity polypeptide that is capable of binding the peptide tag, thereby recruiting the DNA-dependent DNA polymerase to the sequence-specific DNA binding protein.

17. The method of claim 1, wherein the DNA-dependent DNA polymerase is fused to a peptide tag and the sequence-specific DNA binding protein is fused to an affinity polypeptide that is capable of binding the peptide tag, thereby recruiting the DNA-dependent DNA polymerase to the sequence-specific DNA binding protein.

18. The method of claim 17, wherein the peptide tag comprises a GCN4 peptide tag, a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope.

19. A first complex comprising:
(a) a first sequence-specific DNA binding protein that is capable of binding to a first site on a target nucleic acid; and
(b) a first DNA-dependent DNA polymerase.

20. A second complex comprising:
(a) a second sequence-specific DNA binding protein that is capable of binding to a second site on a target nucleic acid; and
(b) a DNA-encoded repair template.


SEQUENCE LISTING (94 sequences)

SEQ ID NO: 1 - protein, 1228 aa, unidentified
  REGION 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 4 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 5 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 6 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 7 - protein, 1206 aa, unidentified
  REGION 1..1206: note = Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 8 - protein, 1233 aa, unidentified
  REGION 1..1233: note = Lachnospiraceae bacterium
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 9 - protein, 1227 aa, unidentified
  REGION 1..1227: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 10 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 11 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 13 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 14 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 15 - protein, 1484 aa, unidentified
  SITE 1073: note = misc_feature - Xaa can be any naturally occurring amino acid
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 16 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 17 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 18 - DNA, 3987 bp, synthetic construct
  misc_feature 1..3987: note = enAsCpf1
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 19 - DNA, 3987 bp, synthetic construct
  misc_feature 1..3987: note = enAsCpf1
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 20 - DNA, 3987 bp, synthetic construct
  misc_feature 1..3987: note = enAsCpf1
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 21 - DNA, 1592 bp, Medicago truncatula
  source 1..1592: mol_type = genomic DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 22 - DNA, 2000 bp, Zea mays
  source 1..2000: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 23 - protein, 24 aa, synthetic construct
  REGION 1..24: note = GCN4 sequence
EELLSKNYHLENEVARLKKGSGSG

SEQ ID NO: 24 - protein, 241 aa, synthetic construct
  REGION 1..241: note = 10-mer GCN4 sequence
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 25 - protein, 277 aa, synthetic construct
  REGION 1..277: note = ScFv antibody
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 26 - DNA, 66 bp, Saccharomyces bayanus
  source 1..66: mol_type = genomic DNA
ttcttgtcgtacttatagatcgctacgttatttcaattttgaaaatctgagtcctgggagtgcgga

SEQ ID NO: 27 - protein, 605 aa, Homo sapiens
MSGWESYYKTEGDEEAEEEQEENLEASGDYKYSGRDSLIFLVDASKAMFESQSEDELTPFDMSIQCIQSVYISKIISSDRDLLAWFYGTEKDKNSVNFKIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPLKEACRAYGLKSGLKKQELLEALTKHFQD

SEQ ID NO: 28 - protein, 482 aa, synthetic construct
  REGION 1..482: note = polypeptide
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 29 - DNA, 10 bp, Methanobacterium thermoautotrophicum
  source 1..10: mol_type = genomic DNA
aatttttgga

SEQ ID NO: 30 - protein, 83 aa, Methanobacterium thermoautotrophicum
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 31 - DNA, 25 bp, Emesvirus zinderi
  source 1..25: mol_type = genomic DNA
gcgcacatgaggatcacccatgtgc

SEQ ID NO: 32 - protein, 116 aa, Emesvirus zinderi
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 33 - DNA, 26 bp, Pepevirus rubrum
  source 1..26: mol_type = genomic DNA
ataaggagtttatatggaaaccctta

SEQ ID NO: 34 - protein, 127 aa, Pepevirus rubrum
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 35 - DNA, 19 bp, Shigella phage
  source 1..19: mol_type = genomic DNA
ctgaatgcctgcgagcatc

SEQ ID NO: 36 - protein, 62 aa, Shigella phage
MKSIRCKNCNKLLFKADSFDHIEIRCPRCKRHIIMLNACEHPTEKHCGKREKITHSDETVRY

SEQ ID NO: 37-39 - skipped (3 entries)

SEQ ID NO: 40 - DNA, 15 bp, unidentified
  misc_feature 1..15: note = Porcine circovirus
  source 1..15: mol_type = genomic DNA
aagtattaccagaaa

SEQ ID NO: 41 - DNA, 15 bp, unidentified
  misc_feature 1..15: note = Duck circovirus
  source 1..15: mol_type = genomic DNA
aagtattaccagaaa

SEQ ID NO: 42 - DNA, 15 bp, Fava bean necrosis yellows virus
  source 1..15: mol_type = genomic DNA
aagtattaccagaaa

SEQ ID NO: 43 - DNA, 23 bp, Streptococcus agalactiae
  source 1..23: mol_type = genomic DNA
tgcttccgtactacgacccccca

SEQ ID NO: 44 - DNA, 23 bp, Fructobacillus tropaeoli
  source 1..23: mol_type = genomic DNA
tgcttccgtactacgacccccca

SEQ ID NO: 45 - DNA, 22 bp, Escherichia coli
  source 1..22: mol_type = genomic DNA
tttgcgtggggtgtggtgcttt

SEQ ID NO: 46 - DNA, 38 bp, Escherichia coli
  source 1..38: mol_type = genomic DNA
ccagtttctcgaagagaaaccggtaagtgcaccctccc

SEQ ID NO: 47 - DNA, 45 bp, Staphylococcus aureus
  source 1..45: mol_type = genomic DNA
acgcgaacggaacgttcgcataagtgcgcccttacgggatttaac

SEQ ID NO: 48 - protein, 2286 aa, Homo sapiens
MSLRSGGRRRADPGADGEASRDDGATSSVSALKRLERSQWTDKMDLRFGFERLKEPGEKTGWLINMHPTEILDEDKRLGSAVDYYFIQDDGSRFKVALPYKPYFYIATRKGCEREVSSFLSKKFQGKIAKVETVPKEDLDLPNHLVGLKRNYIRLSFHTVEDLVKVRKEISPAVKKNREQDHASDAYTALLSSVLQRGGVITDEEETSKKIADQLDNIVDMREYDVPYHIRLSIDLKIHVAHWYNVRYRGNAFPVEITRRDDLVERPDPVVLAFDIETTKLPLKFPDAETDQIMMISYMIDGQGYLITNREIVSEDIEDFEFTPKPEYEGPFCVFNEPDEAHLIQRWFEHVQETKPTIMVTYNGDFFDWPFVEARAAVHGLSMQQEIGFQKDSQGEYKAPQCIHMDCLRWVKRDSYLPVGSHNLKAAAKAKLGYDPVELDPEDMCRMATEQPQTLATYSVSDAVATYYLYMKYVHPFIFALCTIIPMEPDEVLRKGSGTLCEALLMVQAFHANIIFPNKQEQEFNKLTDDGHVLDSETYVGGHVEALESGVFRSDIPCRFRMNPAAFDFLLQRVEKTLRHALEEEEKVPVEQVTNFEEVCDEIKSKLASLKDVPSRIECPLIYHLDVGAMYPNIILTNRLQPSAMVDEATCAACDFNKPGANCQRKMAWQWRGEFMPASRSEYHRIQHQLESEKFPPLFPEGPARAFHELSREEQAKYEKRRLADYCRKAYKKIHITKVEERLTTICQRENSFYVDTVRAFRDRRYEFKGLHKVWKKKLSAAVEVGDAAEVKRCKNMEVLYDSLQLAHKCILNSFYGYVMRKGARWYSMEMAGIVCFTGANIITQARELIEQIGRPLELDTDGIWCVLPNSFPENFVFKTTNVKKPKVTISYPGAMLNIMVKEGFTNDQYQELAEPSSLTYVTRSENSIFFEVDGPYLAMILPASKEEGKKLKKRYAVFNEDGSLAELKGFEVKRRGELQLIKIFQSSVFEAFLKGSTLEEVYGSVAKVADYWLDVLYSKAANMPDSELFELISENRSMSRKLEDYGEQKSTSISTAKRLAEFLGDQMVKDAGLSCRYIISRKPEGSPVTERAIPLAIFQAEPTVRKHFLRKWLKSSSLQDFDIRAILDWDYYIERLGSAIQKIITIPAALQQVKNPVPRVKHPDWLHKKLLEKNDVYKQKKISELFTLEGRRQVTMAEASEDSPRPSAPDMEDFGLVKLPHPAAPVTVKRKRVLWESQEESQDLTPTVPWQEILGQPPALGTSQEEWLVWLRFHKKKWQLQARQRLARRKRQRLESAEGVLRPGAIRDGPATGLGSFLRRTARSILDLPWQIVQISETSQAGLFRLWALVGSDLHCIRLSIPRVFYVNQRVAKAEEGASYRKVNRVLPRSNMVYNLYEYSVPEDMYQEHINEINAELSAPDIEGVYETQVPLLFRALVHLGCVCVVNKQLVRHLSGWEAETFALEHLEMRSLAQFSYLEPGSIRHIYLYHHAQAHKALFGIFIPSQRRASVFVLDTVRSNQMPSLGALYSAEHGLLLEKVGPELLPPPKHTFEVRAETDLKTICRAIQRFLLAYKEERRGPTLIAVQSSWELKRLASEIPVLEEFPLVPICVADKINYGVLDWQRHGARRMIRHYLNLDTCLSQAFEMSRYFHIPIGNLPEDISTFGSDLFFARHLQRHNHLLWLSPTARPDLGGKEADDNCLVMEFDDQATVEINSSGCYSTVCVELDLQNLAVNTILQSHHVNDMEGADSMGISFDVIQQASLEDMITGGQAASAPASYDETALCSNTFRILKSMVVGWVKEITQYHNIYADNQVMHFYRWLRSPSSLLHDPALHRTLHNMMKKLFLQLIAEFKRLGSSVIYANFNRIILCTKKRRVEDAIAYVEYITSSIHSKETFHSLTISFSRCWEFLLWMDPSNYGGIKGKVSSRIHCGLQDSQKAGGAEDEQENEDDEEERDGEEEEEAEESNVEDLLENNWNILQFLPQAASCQNYFLMIVSAYIVAVYHCMKDGLRRSAPGSTPVRRRGASQLSQEAEGAVGALPGMITFSQDYVANELTQSFFTITQKIQKKVTGSRNSTELSEMFPVLPGSHLLLNNPALEFIKYVCKVLSLDTNITNQVNKLNRDLLRLVDVGEFSEEAQFRDPCRSYVLPEVICRSCNFCRDLDLCKDSSFSEDGAVLPQWLCSNCQAPYDSSAIEMTLVEVLQKKLMAFTLQDLVCLKCRGVKETSMPVYCSCAGDFALTIHTQVFMEQIGIFRNIAQHYGMSYLLETLEWLLQKNPQLGH

SEQ ID NO: 49 - protein, 1462 aa, Homo sapiens
MAPVHGDDSLSDSGSFVSSRARREKKSKKGRQEALERLKKAKAGEKYKYEVEDFTGVYEEVDEEQYSKLVQARQDDDWIVDDDGIGYVEDGREIFDDDLEDDALDADEKGKDGKARNKDKRNVKKLAVTKPNNIKSMFIACAGKKTADKAVDLSKDGLLGDILQDLNTETPQITPPPVMILKKKRSIGASPNPFSVHTATAVPSGKIASPVSRKEPPLTPVPLKRAEFAGDDVQVESTEEEQESGAMEFEDGDFDEPMEVEEVDLEPMAAKAWDKESEPAEEVKQEADSGKGTVSYLGSFLPDVSCWDIDQEGDSSFSVQEVQVDSSHLPLVKGADEEQVFHFYWLDAYEDQYNQPGVVFLFGKVWIESAETHVSCCVMVKNIERTLYFLPREMKIDLNTGKETGTPISMKDVYEEFDEKIATKYKIMKFKSKPVEKNYAFEIPDVPEKSEYLEVKYSAEMPQLPQDLKGETFSHVFGTNTSSLELFLMNRKIKGPCWLEVKSPQLLNQPVSWCKVEAMALKPDLVNVIKDVSPPPLVVMAFSMKTMQNAKNHQNEIIAMAALVHHSFALDKAAPKPPFQSHFCVVSKPKDCIFPYAFKEVIEKKNVKVEVAATERTLLGFFLAKVHKIDPDIIVGHNIYGFELEVLLQRINVCKAPHWSKIGRLKRSNMPKLGGRSGFGERNATCGRMICDVEISAKELIRCKSYHLSELVQQILKTERVVIPMENIQNMYSESSQLLYLLEHTWKDAKFILQIMCELNVLPLALQITNIAGNIMSRTLMGGRSERNEFLLLHAFYENNYIVPDKQIFRKPQQKLGDEDEEIDGDTNKYKKGRKKAAYAGGLVLDPKVGFYDKFILLLDFNSLYPSIIQEFNICFTTVQRVASEAQKVTEDGEQEQIPELPDPSLEMGILPREIRKLVERRKQVKQLMKQQDLNPDLILQYDIRQKALKLTANSMYGCLGFSYSRFYAKPLAALVTYKGREILMHTKEMVQKMNLEVIYGDTDSIMINTNSTNLEEVFKLGNKVKSEVNKLYKLLEIDIDGVFKSLLLLKKKKYAALVVEPTSDGNYVTKQELKGLDIVRRDWCDLAKDTGNFVIGQILSDQSRDTIVENIQKRLIEIGENVLNGSVPVSQFEINKALTKDPQDYPDKKSLPHVHVALWINSQGGRKVKAGDTVSYVICQDGSNLTASQRAYAPEQLQKQDNLTIDTQYYLAQQIHPVVARICEPIDGIDAVLIATWLGLDPTQFRVHHYHKDEENDALLGGPAQLTDEEKYRDCERFKCPCPTCGTENIYDNVFDGSGTDMEPSLYRCSNIDCKASPLTFTVQLSNKLIMDIRRFIKKYYDGWLICEEPTCRNRTRHLPLQFSRTGPLCPACMKATLQPEYSDKSLYTQLCFYRYIFDAECALEKLTTDHEKDKLKKQFFTPKVLQDYRKLKNTAEQFLSRSGYSEVNLSKLFAGCAVKS

SEQ ID NO: 50 - protein, 335 aa, Homo sapiens
MSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSE

SEQ ID NO: 51 - protein, 1239 aa, Homo sapiens
MSRLLWRKVAGATVGPGPVPAPGRWVSSSVPASDPSDGQRRRQQQQQQQQQQQQQPQQPQVLSSEGGQLRHNPLDIQMLSRGLHEQIFGQGGEMPGEAAVRRSVEHLQKHGLWGQPAVPLPDVELRLPPLYGDNLDQHFRLLAQKQSLPYLEAANLLLQAQLPPKPPAWAWAEGWTRYGPEGEAVPVAIPEERALVFDVEVCLAEGTCPTLAVAISPSAWYSWCSQRLVEERYSWTSQLSPADLIPLEVPTGASSPTQRDWQEQLVVGHNVSFDRAHIREQYLIQGSRMRFLDTMSMHMAISGLSSFQRSLWIAAKQGKHKVQPPTKQGQKSQRKARRGPAISSWDWLDISSVNSLAEVHRLYVGGPPLEKEPRELFVKGTMKDIRENFQDLMQYCAQDVWATHEVFQQQLPLFLERCPHPVTLAGMLEMGVSYLPVNQNWERYLAEAQGTYEELQREMKKSLMDLANDACQLLSGERYKEDPWLWDLEWDLQEFKQKKAKKVKKEPATASKLPIEGAGAPGDPMDQEDLGPCSEEEEFQQDVMARACLQKLKGTTELLPKRPQHLPGHPGWYRKLCPRLDDPAWTPGPSLLSLQMRVTPKLMALTWDGFPLHYSERHGWGYLVPGRRDNLAKLPTGTTLESAGVVCPYRAIESLYRKHCLEQGKQQLMPQEAGLAEEFLLTDNSAIWQTVEELDYLEVEAEAKMENLRAAVPGQPLALTARGGPKDTQPSYHHGNGPYNDVDIPGCWFFKLPHKDGNSCNVGSPFAKDFLPKMEDGTLQAGPGGASGPRALEINKMISFWRNAHKRISSQMVVWLPRSALPRAVIRHPDYDEEGLYGAILPQVVTAGTITRRAVEPTWLTASNARPDRVGSELKAMVQAPPGYTLVGADVDSQELWIAAVLGDAHFAGMHGCTAFGWMTLQGRKSRGTDLHSKTATTVGISREHAKIFNYGRIYGAGQPFAERLLMQFNHRLTQQEAAEKAQQMYAATKGLRWYRLSDEGEWLVRELNLPVDRTEGGWISLQDLRKVQRETARKSQWKKWEVVAERAWKGGTESEMFNKLESIATSDIPRTPVLGCCISRALEPSAVQEEFMTSRVNWVVQSSAVDYLHLMLVAMKWLFEEFAIDGRFCISIHDEVRYLVREEDRYRAALALQITNLLTRCMFAYKLGLNDLPQSVAFFSAVDIDRCLRKEVTMDCKTPSNPTGMERRYGIPQGEALDIYQIIELTKGSLEKRSQPGP

SEQ ID NO: 52 - protein, 1187 aa, Saccharomyces cerevisiae
MMFGKKKNNGGSSTARYSAGNKYNTLSNNYALSAQQLLNASKIDDIDSMMGFERYVPPQYNGRFDAKDIDQIPGRVGWLTNMHATLVSQETLSSGSNGGGNSNDGERVTTNQGISGVDFYFLDEEGGSFKSTVVYDPYFFIACNDESRVNDVEELVKKYLESCLKSLQIIRKEDLTMDNHLLGLQKTLIKLSFVNSNQLFEARKLLRPILQDNANNNVQRNIYNVAANGSEKVDAKHLIEDIREYDVPYHVRVSIDKDIRVGKWYKVTQQGFIEDTRKIAFADPVVMAFDIETTKPPLKFPDSAVDQIMMISYMIDGEGFLITNREIISEDIEDFEYTPKPEYPGFFTIFNENDEVALLQRFFEHIRDVRPTVISTFNGDFFDWPFIHNRSKIHGLDMFDEIGFAPDAEGEYKSSYCSHMDCFRWVKRDSYLPQGSQGLKAVTQSKLGYNPIELDPELMTPYAFEKPQHLSEYSVSDAVATYYLYMKYVHPFIFSLCTIIPLNPDETLRKGTGTLCEMLLMVQAYQHNILLPNKHTDPIERFYDGHLLESETYVGGHVESLEAGVFRSDLKNEFKIDPSAIDELLQELPEALKFSVEVENKSSVDKVTNFEEIKNQITQKLLELKENNIRNELPLIYHVDVASMYPNIMTTNRLQPDSIKAERDCASCDFNRPGKTCARKLKWAWRGEFFPSKMDEYNMIKRALQNETFPNKNKFSKKKVLTFDELSYADQVIHIKKRLTEYSRKVYHRVKVSEIVEREAIVCQRENPFYVDTVKSFRDRRYEFKGLAKTWKGNLSKIDPSDKHARDEAKKMIVLYDSLQLAHKVILNSFYGYVMRKGSRWYSMEMAGITCLTGATIIQMARALVERVGRPLELDTDGIWCILPKSFPETYFFTLENGKKLYLSYPCSMLNYRVHQKFTNHQYQELKDPLNYIYETHSENTIFFEVDGPYKAMILPSSKEEGKGIKKRYAVFNEDGSLAELKGFELKRRGELQLIKNFQSDIFKVFLEGDTLEGCYSAVASVCNRWLDVLDSHGLMLEDEDLVSLICENRSMSKTLKEYEGQKSTSITTARRLGDFLGEDMVKDKGLQCKYIISSKPFNAPVTERAIPVAIFSADIPIKRSFLRRWTLDPSLEDLDIRTIIDWGYYRERLGSAIQKIITIPAALQGVSNPVPRVEHPDWLKRKIATK

SEQ ID NO: 53 - protein, 632 aa, Saccharomyces cerevisiae
MSKFTWKELIQLGSPSKAYESSLACIAHIDMNAFFAQVEQMRCGLSKEDPVVCVQWNSIIAVSYAARKYGISRMDTIQEALKKCSNLIPIHTAVFKKGEDFWQYHDGCGSWVQDPAKQISVEDHKVSLEPYRRESRKALKIFKSACDLVERASIDEVFLDLGRICFNMLMFDNEYELTGDLKLKDALSNIREAFIGGNYDINSHLPLIPEKIKSLKFEGDVFNPEGRDLITDWDDVILALGSQVCKGIRDSIKDILGYTTSCGLSSTKNVCKLASNYKKPDAQTIVKNDCLLDFLDCGKFEITSFWTLGGVLGKELIDVLDLPHENSIKHIRETWPDNAGQLKEFLDAKVKQSDYDRSTSNIDPLKTADLAEKLFKLSRGRYGLPLSSRPVVKSMMSNKNLRGKSCNSIVDCISWLEVFCAELTSRIQDLEQEYNKIVIPRTVSISLKTKSYEVYRKSGPVAYKGINFQSHELLKVGIKFVTDLDIKGKNKSYYPLTKLSMTITNFDIIDLQKTVVDMFGNQVHTFKSSAGKEDEEKTTSSKADEKTPKLECCKYQVTFTDQKALQEHADYHLALKLSEGLNGAEESSKNLSFGEKRLLFSRKRPNSQHTATPQKKQVTSSKNILSFFTRKK

SEQ ID NO: 54 - protein, 928 aa, Escherichia coli [sequence omitted]

SEQ ID NO: 55 - protein, 783 aa, Escherichia coli
MAQAGFILTRHWRDTPQGTEVSFWLATDNGPLQVTLAPQESVAFIPADQVPRAQHILQGEQGFRLTPLALKDFHRQPVYGLYCRAHRQLMNYEKRLREGGVTVYEADVRPPERYLMERFITSPVWVEGDMHNGTIVNARLKPHPDYRPPLKWVSIDIETTRHGELYCIGLEGCGQRIVYMLGPENGDASSLDFELEYVASRPQLLEKLNAWFANYDPDVIIGWNVVQFDLRMLQKHAERYRLPLRLGRDNSELEWREHGFKNGVFFAQAKGRLIIDGIEALKSAFWNFSSFSLETVAQELLGEGKSIDNPWDRMDEIDRRFAEDKPALATYNLKDCELVTQIFHKTEIMPFLLERATVNGLPVDRHGGSVAAFGHLYFPRMHRAGYVAPNLGEVPPHASPGGYVMDSRPGLYDSVLVLDYKSLYPSIIRTFLIDPVGLVEGMAQPDPEHSTEGFLDAWFSREKHCLPEIVTNIWHGRDEAKRQGNKPLSQALKIIMNAFYGVLGTTACRFFDPRLASSITMRGHQIMRQTKALIEAQGYDVIYGDTDSTFVWLKGAHSEEEAAKIGRALVQHVNAWWAETLQKQRLTSALELEYETHFCRFLMPTIRGADTGSKKRYAGLIQEGDKQRMVFKGLETVRTDWTPLAQQFQQELYLRIFRNEPYQEYVRETIDKLMAGELDARLVYRKRLRRPLSEYQRNVPPHVRAARLADEENQKRGRPLQYQNRGTIKYVWTTNGPEPLDYQRSPLDYEHYLTRQLQPVAEGILPFIEDNFATLMTGQLGLF

SEQ ID NO: 56 - protein, 1160 aa, Escherichia coli
MSEPRFVHLRVHSDYSMIDGLAKTAPLVKKAAALGMPALAITDFTNLCGLVKFYGAGHGAGIKPIVGADFNVQCDLLGDELTHLTVLAANNTGYQNLTLLISKAYQRGYGAAGPIIDRDWLIELNEGLILLSGGRMGDVGRSLLRGNSALVDECVAFYEEHFPDRYFLELIRTGRPDEESYLHAAVELAEARGLPVVATNDVRFIDSSDFDAHEIRVAIHDGFTLDDPKRPRNYSPQQYMRSEEEMCELFADIPEALANTVEIAKRCNVTVRLGEYFLPQFPTGDMSTEDYLVKRAKEGLEERLAFLFPDEEERLKRRPEYDERLETELQVINQMGFPGYFLIVMEFIQWSKDNGVPVGPGRGSGAGSLVAYALKITDLDPLEFDLLFERFLNPERVSMPDFDVDFCMEKRDQVIEHVADMYGRDAVSQIITFGTMAAKAVIRDVGRVLGHPYGFVDRISKLIPPDPGMTLAKAFEAEPQLPEIYEADEEVKALIDMARKLEGVTRNAGKHAGGVVIAPTKITDFAPLYCDEEGKHPVTQFDKSDVEYAGLVKFDFLGLRTLTIINWALEMINKRRAKNGEPPLDIAAIPLDDKKSFDMLQRSETTAVFQLESRGMKDLIKRLQPDCFEDMIALVALFRPGPLQSGMVDNFIDRKHGREEISYPDVQWQHESLKPVLEPTYGIILYQEQVMQIAQVLSGYTLGGADMLRRAMGKKKPEEMAKQRSVFAEGAEKNGINAELAMKIFDLVEKFAGYGFNKSHSAAYALVSYQTLWLKAHYPAEFMAAVMTADMDNTEKVVGLVDECWRMGLKILPPDINSGLYHFHVNDDGEIVYGIGAIKGVGEGPIEAIIEARNKGGYFRELFDLCARTDTKKLNRRVLEKLIMSGAFDRLGPHRAALMNSLGDALKAADQHAKAEAIGQADMFGVLAEEPEQIEQSYASCQPWPEQVVLDGERETLGLYLTGHPINQYLKEIERYVGGVRLKDMHPTERGKVITAAGLVVAARVMVTKRGNRIGICTLDDRSGRLEVMLFTDALDKYQQLLEKDRILIVSGQVSFDDFSGGLKMTAREVMDIDEAREKYARGLAISLTDRQIDDQLLNRLRQSLEPHRSGTIPVHLYYQRADARARLRFGATWRVSPSDRLLNDLRGLIGSEQVELEFD

SEQ ID NO: 57 - protein, 775 aa, Thermococcus guaymasensis
MILDTDYITENGKPVIRVFKKENGEFKIEYDRTFEPYFYALLKDDSAIEDVKKVTAKRHRTIVKVKRAEKVQKKFLRRPIEVWKLYFNHPQDVPAIRDKIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVKEKDPDVLITYNGDNFDFAYLKKRCEKLGIKFTLGRDGSEPKIQRMGDRFAVEVKGRIHFDLYPVIRRTINLPTYTLEAVYEAVFGKPKEKVYAEEITEAWESGEGLERVARYSMEDAKVTYELGREFFPMEAQLSRLIGQSLWDVSRSSTGNLVEWFLLRKAYERNELAPNKPDERELARRRESYAGGYVKEPERGLWENIVYLDFMSLYPSIIITHNVSPDTLNREGCREYDVAPEVGHKFCKDFPGFIPSLLGDLLEERQKIKRKMKATIDPLEKKILDYRQRAIKILANSYYGYYGYAKARWYCRECAESVTAWGREYIETTIREIEEKFGFKVLYADTDGFFATIPGANAETVKKKAREFLKYINAKLPGLLELEYEGFYLRGFFVTKKKYALIDEEGKITTRGLEIVRRDWSEIAKETQARVLEAILKHGDIEEAVRIVKEVTEKLSKYEVPPEKLVIHEQITRDLRDYKATGPHVAVAKRLAARGVKIRPGTVISYIVLRGSGRIGDRAIPADEFDPTKHKYDAEYYIENQVLPAVERILRAFGYRKEDLRYQKTKQVGLGAWLKVKGRK

SEQ ID NO: 58 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 59 - DNA, 4101 bp, synthetic construct
  misc_feature 1..4101: note = Cas9
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 60 - DNA, 4101 bp, synthetic construct
  misc_feature 1..4101: note = nCas9
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctggccgacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctgccctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaaggcccctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 61 - DNA, 4101 bp, synthetic construct
  misc_feature 1..4101: note = nCas9(D10A)
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 62 - DNA, 4101 bp, synthetic construct
  misc_feature 1..4101: note = nCas9(H840A)
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 63 - DNA, 1761 bp, Escherichia coli
  source 1..1761: mol_type = genomic DNA
atgacaaaaacatctaaacttgacgcacttagggctgctacttcacgtgaagacttggctaaaattttagatattaagttggtatttttaactaacgttctatatagaatcggctcggataatcaatacactcaatttacaataccgaagaaaggaaaaggggtaaggactatttctgcacctacagaccggttgaaggacatccaacgaagaatatgtgacttactttctgattgtagagatgagatctttgctataaggaaaattagtaacaactattcctttggttttgagaggggaaaatcaataatcctaaatgcttataagcatagaggcaaacaaataatattaaatatagatcttaaggatttttttgaaagctttaattttggacgagttagaggatattttctttccaatcaggattttttattaaatcctgtggtggcaacgacacttgcaaaagctgcatgctataatggaaccctcccccaaggaagtccatgttctcctattatctcaaatctaatttgcaatattatggatatgagattagctaagctggctaaaaaatatggatgtacttatagcagatatgctgatgatataacaatttctacaaataaaaatacatttccgttagaaatggctactgtgcaacctgaaggggttgttttgggaaaagttttggtaaaagaaatagaaaactctggattcgaaataaatgattcaaagactaggcttacgtataagacatcaaggcaagaagtaacgggacttacagttaacagaatcgttaatattgatagatgttattataaaaaaactcgggcgttggcacatgctttgtatcgtacaggtgaatataaagtgccagatgaaaatggtgttttagtttcaggaggtctggataaacttgaggggatgtttggttttattgatcaagttgataagtttaacaatataaagaaaaaactgaacaagcaacctgatagatatgtattgactaatgcgactttgcatggttttaaattaaagttgaatgcgcgagaaaaagcatatagtaaatttatttactataaattttttcatggcaacacctgtcctacgataattacagaagggaagactgatcggatatatttgaaggctgctttgcattctttggagacatcatatcctgagttgtttagagaaaaaacagatagtaaaaagaaagaaataaatcttaatatatttaaatctaatgaaaagaccaaatattttttagatctttctgggggaactgcagatctgaaaaaatttgtagagcgttataaaaataattatgcttcttattatggttctgttccaaaacagccagtgattatggttcttgataatgatacaggtccaagcgatttacttaattttctgcgcaataaagttaaaagctgcccagacgatgtaactgaaatgagaaagatgaaatatattcatgttttctataatttatatatagttctcacaccattgagtccttccggcgaacaaacttcaatggaggatcttttccctaaagatattttagatatcaagattgatggtaagaaattcaacaaaaataatgatggagactcaaaaacggaatatgggaagcatattttttccatgagggttgttagagataaaaagcggaaaatagattttaaggcattttgttgtatttttgatgctataaaagatataaaggaacattataaattaatgttaaatagctaa

SEQ ID NO: 64 - DNA, 132 bp, Escherichia coli
  source 1..132: mol_type = genomic DNA
cacgcatgtaggcagatttgttggttgtgaatcgcaaccagtggccttaatggcaggaggaatcgcctccctaaaatccttgattcagagctatacggcaggtgtgctgtgcgaaggagtgcctgcatgcgt

SEQ ID NO: 65 - DNA, 99 bp, Escherichia coli
  source 1..99: mol_type = genomic DNA
atgaaatcgcatgatcgattgaggatcgtctttgctcagatccgccagaactggcggcttttgctcatgttatgcatgtgcatgaaaaccactgcataa

SEQ ID NO: 66 - DNA, 170 bp, Escherichia coli
  source 1..170: mol_type = genomic DNA
atgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgca

SEQ ID NO: 67 - DNA, 960 bp, Escherichia coli
  source 1..960: mol_type = genomic DNA
atggatgctacccggacaacccttctggcgctcgatttgttcggctcgccgggctggagcgccgataaagaaatacagcgactgcatgcgctcagtaatcatgccggacgccattaccgacgcattattctttctaaacgccacggtggtcagcggctggtgttagcccctgattacttgctcaaaaccgtacagcgcaacattcttaagaacgtcctttcacaatttccgctttccccttttgctacagcctaccgaccaggttgcccaatcgtcagcaacgcgcagccacactgccaacagccgcagatcctgaaactcgatatcgaaaactttttcgatagcattagctggttacaggtctggcgtgtgtttcgccaggcccagttgccacgtaatgtggtaaccatgctgacctggatttgttgttataacgacgcgttaccgcagggggcaccaacttcgccagccatttccaatcttgtgatgcgccgttttgatgaacgcataggggaatggtgtcaggctcggggaattacctacacccgctactgcgatgacatgaccttttcaggtcacttcaatgcccgccaggttaaaaataaagtgtgcggattgttagcggagctgggcctgagcctcaataaacgcaaaggctgcctgatagctgcctgtaagcgccagcaagtaaccgggattgttgttaatcacaagccacagcttgcccgtgaagcgcgccgggcgctgcgtcaggaggtgcatttgtgccaaaaatatggcgttatttcgcatcttagtcatcgtggtgaacttgatccttctggcgatctccacgcacaggcaacggcgtatctttatgctttgcagggaagaataaactggttattgcaaatcaaccctgaggatgaggcctttcaacaggcgagagagagtgtaaagcgaatgctggttgcatggtaa

SEQ ID NO: 68 - DNA, 176 bp, Escherichia coli
  source 1..176: mol_type = genomic DNA
cgccagcagtggcaatagcgtttccggccttttgtgccgggagggtcggcgagtcgctgacttaacgccagtagtatgtccatatacccaaagtcgcttcattgtacctgagtacgcttcgcgtacgtcgcgctgacgcgctcagtacagttacgcgccttcgggatggtttaatg

SEQ ID NO: 69 - DNA, 1458 bp, Myxococcus xanthus
  source 1..1458: mol_type = genomic DNA
atgaccgccaggctggacccgttcgtccccgcagcttcgccgcaggccgtgcccacgcccgagctcaccgctccgtcgtcagacgcggccgcgaagcgtgaagcccgccggctcgcgcacgaagcgttgctcgtccgcgcgaaggccatcgacgaagcgggcggcgccgacgactgggtgcaggcgcagctcgtctccaagggcctcgcggtggaggacctggacttctccagcgcctccgagaaggacaagaaggcctggaaggagaagaagaaggccgaggccaccgagcgccgcgcgctgaagcgtcaggcgcacgaggcgtggaaggccacgcacgtgggccacctgggcgcgggcgtgcactgggcggaggaccgcctggccgacgcgttcgacgtgccccaccgcgaggagcgcgcccgggccaacggcctgacggagctggactcggcggaggcgctggccaaggcgctggggctgagcgtgtccaagctgcgctggttcgcgttccaccgcgaggtggacacggccacgcactacgtgagctggacgattccgaagcgggacggcagcaagcgcacgattacgtcccccaagcctgagctgaaggcagcgcagcgctgggtgctgtccaacgtcgtggagcggctgccggtgcacggcgcggcgcacggcttcgtggcgggacgctccatcctcaccaacgcgctggcccaccagggcgcggacgtggtggtgaaggtggacctcaaggacttcttcccctccgtcacctggcgccgggtgaagggcctgttgcgcaagggcggcctgcgggagggcacgtccacgctgctgtcgctgctctccacggaagcgccgcgggaggcggtgcagttccggggcaagctgctgcacgtggccaagggcccgcgcgcgctgccccagggcgcgcccacgtcgccgggcatcaccaacgcgctgtgcctgaagctggacaagcggctgtccgcgctcgcgaagcggctgggcttcacgtacacgcgctacgcggacgacctgaccttctcgtggacgaaggcgaagcagcccaagccgcggcggacgcagcgtcccccggtggcggtgctgctgtctcgcgtgcaggaagtggtggaggcggagggcttccgcgtgcacccggacaagacgcgcgtggcgcgcaagggcacgcggcagcgggtgacggggctggtcgtgaatgcggcgggcaaggacgcgccggcggcccgagtcccgcgcgacgtggtgcgccagctccgcgccgccatccacaaccggaagaagggcaagccgggccgcgagggcgagtcgctggagcagctcaagggcatggccgccttcatccacatgacggacccggccaagggccgcgccttcctggctcagctcacggagctggagtccacggcgagcgcggctccgcaggcggagtga

SEQ ID NO: 70 - DNA, 231 bp, Myxococcus xanthus
  source 1..231: mol_type = genomic DNA
agaggtccggagtgcatcagcctgagcgcctcgagcggcggagcggcgttgcgccgctccggttggaatgcaggacactctccgcaaggtagcctgttcttggctctctccctcctaggcactacggccagggtgggtagcggagccaacgacgcgaccgccgtttacccaccccggccgtagtgcctaggaggggagagccggtgaggctaccgtgccccaggtaagatg

SEQ ID NO: 71 - DNA, 334 bp, synthetic construct
  misc_feature 1..334: note = Chimeric guide nucleic acid sequences (guide DNA)
  source 1..334: mol_type = other DNA
cacgcatgtaggcagatttgttggttgtgaatcgcaaccagtggccttaatggcaggaggaatcgcctccagagtcgccgtctccaaggtgaaagcggaagtagggccttcgcgcacctcatggaatcccttctgcagcacctagatcgcttttctgaactcctagcagtatctagcactacctacgtcagcacctgggaccccgcggtgtgctgtgcgaaggagtgcctgcatgcgtggaatcccttctgcagcaccgttttagagctagaaatagcaagttaaaataaggctagtccgttatcaacttgaaaaagtggcaccgagtcggtgc

SEQ ID NO: 72 - DNA, 361 bp, synthetic construct
  misc_feature 1..361: note = Chimeric guide nucleic acid sequences (guide DNA)
  source 1..361: mol_type = other DNA
atgcgcacccttagcgagaggtttatcattaaggtcaacctctggatgttgtttcggcatcctgcattgaatctgagttactgtctgttttcctagagtcgccgtctccaaggtgaaagcggaagtagggccttcgcgcacctcatggaatcccttctgcagcacctagatcgcttttctgaactcctagcagtatctagcactacctacgtcagcacctgggaccccgccaggaaacccgttttttctgacgtaagggtgcgcaggaatcccttctgcagcaccgttttagagctagaaatagcaagttaaaataaggctagtccgttatcaacttgaaaaagtggcaccgagtcggtgc

SEQ ID NO: 73 - DNA, 345 bp, synthetic construct
  misc_feature 1..345: note = Chimeric guide nucleic acid sequences (guide DNA)
  source 1..345: mol_type = other DNA
gccagcagtggcaatagcgtttccggccttttgtgccgggagggtcggcgagtcgctgacttaacgccagtagtatgtccatatacccaagagtcgccgtctccaaggtgaaagcggaagtagggccttcgcgcacctcatggaatcccttctgcagcacctagatcgcttttctgaactcctagcagtatctagcactacctacgtcagcacctgggaccccgcgggatggtttaatggtattgccgcggaatcccttctgcagcaccgttttagagctagaaatagcaagttaaaataaggctagtccgttatcaacttgaaaaagtggcaccgagtcggtgc

SEQ ID NO: 74 - DNA, 364 bp, synthetic construct
  misc_feature 1..364: note = Chimeric guide nucleic acid sequences (guide DNA)
  source 1..364: mol_type = other DNA
agaggtccggagtgcatcagcctgagcgcctcgagcggcggagcggcgttgcgccgctccggttggaatgcaggacactctccgcaaggtagagtcgccgtctccaaggtgaaagcggaagtagggccttcgcgcacctcatggaatcccttctgcagcacctagatcgcttttctgaactcctagcagtatctagcactacctacgtcagcacctgggaccccgctgaggctaccgtgccccaggtaagatggtggtgctttcccggggaatcccttctgcagcaccgttttagagctagaaatagcaagttaaaataaggctagtccgttatcaacttgaaaaagtggcaccgagtcggtgc

SEQ ID NO: 75 - protein, 1244 aa, synthetic construct
  REGION 1..1244: note = Cas12a RNP protein sequence
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHPKKKRKVLEHHHHHH

SEQ ID NO: 76 - RNA, 23 nt, synthetic construct
  misc_feature 1..23: note = PWsp137 Target Sequence
  source 1..23: mol_type = other RNA
cctcactcctgctcggtgaattt

SEQ ID NO: 77 - RNA, 43 nt, synthetic construct
  misc_feature 1..43: note = PWsp137 crRNA
  source 1..43: mol_type = other RNA
aatttctactaagtgtagatcctcactcctgctcggtgaattt

SEQ ID NO: 78 - RNA, 103 nt, synthetic construct
  source 1..103: mol_type = other RNA
  modified_base 45: mod_base = OTHER; note = thymine
  modified_base 53: mod_base = OTHER; note = thymine
  modified_base 59: mod_base = OTHER; note = thymine
  modified_base 63: mod_base = OTHER; note = thymine
  modified_base 66: mod_base = OTHER; note = thymine
  modified_base 69: mod_base = OTHER; note = thymine
  modified_base 73: mod_base = OTHER; note = thymine
  modified_base 77..79: mod_base = OTHER; note = thymine
  modified_base 83: mod_base = OTHER; note = thymine
  modified_base 95: mod_base = OTHER; note = thymine
  modified_base 99: mod_base = OTHER; note = thymine
aatttctactaagtgtagatcctcactcctgctcggtgaatttctggggccgtaaccctcactcctgctcggtgaatttggctcagcaggcacctgcctcagc

SEQ ID NO: 79 - RNA, 115 nt, synthetic construct
  source 1..115: mol_type = other RNA
  modified_base 45: mod_base = OTHER; note = thymine
  modified_base 53: mod_base = OTHER; note = thymine
  modified_base 59: mod_base = OTHER; note = thymine
  modified_base 63: mod_base = OTHER; note = thymine
  modified_base 66: mod_base = OTHER; note = thymine
  modified_base 69: mod_base = OTHER; note = thymine
  modified_base 73: mod_base = OTHER; note = thymine
  modified_base 77..79: mod_base = OTHER; note = thymine
  modified_base 83: mod_base = OTHER; note = thymine
  modified_base 95: mod_base = OTHER; note = thymine
  modified_base 99: mod_base = OTHER; note = thymine
  modified_base 104: mod_base = OTHER; note = thymine
  modified_base 107: mod_base = OTHER; note = thymine
  modified_base 111..112: mod_base = OTHER; note = thymine
aatttctactaagtgtagatcctcactcctgctcggtgaatttctggggccgtaaccctcactcctgctcggtgaatttggctcagcaggcacctgcctcagctgctcacttgag

SEQ ID NO: 80 - RNA, 23 nt, synthetic construct
  misc_feature 1..23: note = PWsp453 Target Sequence
  source 1..23: mol_type = other RNA
tatgagttacaacgaacacctca

SEQ ID NO: 81 - RNA, 43 nt, synthetic construct
  misc_feature 1..43: note = PWsp453 crRNA
  source 1..43: mol_type = other RNA
aatttctactaagtgtagattatgagttacaacgaacacctca

SEQ ID NO: 82 - RNA, 103 nt, synthetic construct
  source 1..103: mol_type = other RNA
  modified_base 49: mod_base = OTHER; note = thymine
  modified_base 53: mod_base = OTHER; note = thymine
  modified_base 57: mod_base = OTHER; note = thymine
  modified_base 59: mod_base = OTHER; note = thymine
  modified_base 63..64: mod_base = OTHER; note = thymine
  modified_base 77: mod_base = OTHER; note = thymine
  modified_base 82: mod_base = OTHER; note = thymine
  modified_base 85: mod_base = OTHER; note = thymine
  modified_base 89: mod_base = OTHER; note = thymine
  modified_base 94: mod_base = OTHER; note = thymine
  modified_base 98: mod_base = OTHER; note = thymine
aatttctactaagtgtagattatgagttacaacgaacacctcaggaactcagtaaatatgagttacaacgaacacctcaggtaatgactaagatgactgccaa

SEQ ID NO: 83 - RNA, 115 nt, synthetic construct
  source 1..115: mol_type = other RNA
  modified_base 49: mod_base = OTHER; note = thymine
  modified_base 53: mod_base = OTHER; note = thymine
  modified_base 57: mod_base = OTHER; note = thymine
  modified_base 59: mod_base = OTHER; note = thymine
  modified_base 63..64: mod_base = OTHER; note = thymine
  modified_base 77: mod_base = OTHER; note = thymine
  modified_base 82: mod_base = OTHER; note = thymine
  modified_base 85: mod_base = OTHER; note = thymine
  modified_base 89: mod_base = OTHER; note = thymine
  modified_base 94: mod_base = OTHER; note = thymine
  modified_base 98: mod_base = OTHER; note = thymine
  modified_base 110: mod_base = OTHER; note = thymine
  modified_base 112: mod_base = OTHER; note = thymine
aatttctactaagtgtagattatgagttacaacgaacacctcaggaactcagtaaatatgagttacaacgaacacctcaggtaatgactaagatgactgccaaggggcatatgag

SEQ ID NO: 84 - RNA, 23 nt, synthetic construct
  misc_feature 1..23: note = PWsp454 Target Sequence
  source 1..23: mol_type = other RNA
cacgtctcatatgccccttggca

SEQ ID NO: 85 - RNA, 43 nt, synthetic construct
  misc_feature 1..43: note = PWsp454 crRNA
  source 1..43: mol_type = other RNA
aatttctactaagtgtagatcacgtctcatatgccccttggca

SEQ ID NO: 86 - RNA, 103 nt, synthetic construct
  source 1..103: mol_type = other RNA
  modified_base 45: mod_base = OTHER; note = thymine
  modified_base 47: mod_base = OTHER; note = thymine
  modified_base 53: mod_base = OTHER; note = thymine
  modified_base 61: mod_base = OTHER; note = thymine
  modified_base 63: mod_base = OTHER; note = thymine
  modified_base 66: mod_base = OTHER; note = thymine
  modified_base 68: mod_base = OTHER; note = thymine
  modified_base 74..75: mod_base = OTHER; note = thymine
  modified_base 81: mod_base = OTHER; note = thymine
  modified_base 84: mod_base = OTHER; note = thymine
  modified_base 86..87: mod_base = OTHER; note = thymine
  modified_base 90: mod_base = OTHER; note = thymine
  modified_base 93..94: mod_base = OTHER; note = thymine
  modified_base 98: mod_base = OTHER; note = thymine
  modified_base 103: mod_base = OTHER; note = thymine
aatttctactaagtgtagatcacgtctcatatgccccttggcagtatcccagtaaacacgtctcatatgccccttggcagtcatcttagtcattacctgaggt

SEQ ID NO: 87 - RNA, 115 nt, synthetic construct
  source 1..115: mol_type = other RNA
  modified_base 45: mod_base = OTHER; note = thymine
  modified_base 47: mod_base = OTHER; note = thymine
  modified_base 53: mod_base = OTHER; note = thymine
  modified_base 61: mod_base = OTHER; note = thymine
  modified_base 63: mod_base = OTHER; note = thymine
  modified_base 66: mod_base = OTHER; note = thymine
  modified_base 68: mod_base = OTHER; note = thymine
  modified_base 74..75: mod_base = OTHER; note = thymine
  modified_base 81: mod_base = OTHER; note = thymine
  modified_base 84: mod_base = OTHER; note = thymine
  modified_base 86..87: mod_base = OTHER; note = thymine
  modified_base 90: mod_base = OTHER; note = thymine
  modified_base 93..94: mod_base = OTHER; note = thymine
  modified_base 98: mod_base = OTHER; note = thymine
  modified_base 103: mod_base = OTHER; note = thymine
  modified_base 105..106: mod_base = OTHER; note = thymine
  modified_base 109..110: mod_base = OTHER; note = thymine
  modified_base 112: mod_base = OTHER; note = thymine
aatttctactaagtgtagatcacgtctcatatgccccttggcagtatcccagtaaacacgtctcatatgccccttggcagtcatcttagtcattacctgaggtgttcgttgtaac

SEQ ID NO: 88 - protein, 579 aa, synthetic construct
  REGION 1..579: note = Klentaq aa
MSGGSKRTADGSEFEPKKKRKVGSSGGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 89 - protein, 800 aa, synthetic construct
  REGION 1..800: note = Therminator aa
MSGGSKRTADGSEFEPKKKRKVGSSGILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 90 - protein, 876 aa, synthetic construct
  REGION 1..876: note = Pfu-Ssod7 aa
MSGGSKRTADGSEFEPKKKRKVGSSGHIEGRHMILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTGGGGATVKFKYKGEEKEVDISKIKKVWRVGKMISFTYDEGGGKTGRGAVSEKDAPKELLQMLEKQKK

SEQ ID NO: 91 - protein, 648 aa, synthetic construct
  REGION 1..648: note = Klenow aa
MSGGSKRTADGSEFEPKKKRKVVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFEPKKKRKV

SEQ ID NO: 92 - protein, 970 aa, Escherichia coli
MSGGSKRTADGSEFEPKKKRKVVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFEPKKKRKV

SEQ ID NO: 93 - protein, 1654 aa, Homo sapiens
MKRTADGSEFESPKKKRKVGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGSRVIEKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGSGGSSGGSSGSETPGTSESATPESSGGSSGGSMSLRSGGRRRADPGADGEASRDDGATSSVSALKRLERSQWTDKMDLRFGFERLKEPGEKTGWLINMHPTEILDEDKRLGSAVDYYFIQDDGSRFKVALPYKPYFYIATRKGCEREVSSFLSKKFQGKIAKVETVPKEDLDLPNHLVGLKRNYIRLSFHTVEDLVKVRKEISPAVKKNREQDHASDAYTALLSSVLQRGGVITDEEETSKKIADQLDNIVDMREYDVPYHIRLSIDLKIHVAHWYNVRYRGNAFPVEITRRDDLVERPDPVVLAFDIETTKLPLKFPDAETDQIMMISYMIDGQGYLITNREIVSEDIEDFEFTPKPEYEGPFCVFNEPDEAHLIQRWFEHVQETKPTIMVTYNGDFFDWPFVEARAAVHGLSMQQEIGFQKDSQGEYKAPQCIHMDCLRWVKRDSYLPVGSHNLKAAAKAKLGYDPVELDPEDMCRMATEQPQTLATYSVSDAVATYYLYMKYVHPFIFALCTIIPMEPDEVLRKGSGTLCEALLMVQAFHANIIFPNKQEQEFNKLTDDGHVLDSETYVGGHVEALESGVFRSDIPCRFRMNPAAFDFLLQRVEKTLRHALEEEEKVPVEQVTNFEEVCDEIKSKLASLKDVPSRIECPLIYHLDVGAMYPNIILTNRLQPSAMVDEATCAACDFNKPGANCQRKMAWQWRGEFMPASRSEYHRIQHQLESEKFPPLFPEGPARAFHELSREEQAKYEKRRLADYCRKAYKKIHITKVEERLTTICQRENSFYVDTVRAFRDRRYEFKGLHKVWKKKLSAAVEVGDAAEVKRCKNMEVLYDSLQLAHKCILNSFYGYVMRKGARWYSMEMAGIVCFTGANIITQARELIEQIGRPLELDTDGIWCVLPNSFPENFVFKTTNVKKPKVTISYPGAMLNIMVKEGFTNDQYQELAEPSSLTYVTRSENSIFFEVDGPYLAMILPASKEEGKKLKKRYAVFNEDGSLAELKGFEVKRRGELQLIKIFQSSVFEAFLKGSTLEEVYGSVAKVADYWLDVLYSKAANMPDSELFELISENRSMSRKLEDYGEQKSTSISTAKRLAEFLGDQMVKDAGLSCRYIISRKPEGSPVTERAIPLAIFQAEPTVRKHFLRKWLKSSSLQDFDIRAILDWDYYIERLGSAIQKIITIPAALQQVKNPVPRVKHPDWLHKKLLEKNDVYKQKKISELFTLEGRRQVTMAEASGGSKRTADGSEFEPKKKRKVGSGATNFSLLKQAGDVEENPGPSGGSPKKKRKV

SEQ ID NO: 94 - protein, 1233 aa, Saccharomyces cerevisiae
MSGGSKRTADGSEFEPKKKRKVGSSGMFGKKKNNGGSSTARYSAGNKYNTLSNNYALSAQQLLNASKIDDIDSMMGFERYVPPQYNGRFDAKDIDQIPGRVGWLTNMHATLVSQETLSSGSNGGGNSNDGERVTTNQGISGVDFYFLDEEGGSFKSTVVYDPYFFIACNDESRVNDVEELVKKYLESCLKSLQIIRKEDLTMDNHLLGLQKTLIKLSFVNSNQLFEARKLLRPILQDNANNNVQRNIYNVAANGSEKVDAKHLIEDIREYDVPYHVRVSIDKDIRVGKWYKVTQQGFIEDTRKIAFADPVVMAFDIETTKPPLKFPDSAVDQIMMISYMIDGEGFLITNREIISEDIEDFEYTPKPEYPGFFTIFNENDEVALLQRFFEHIRDVRPTVISTFNGDFFDWPFIHNRSKIHGLDMFDEIGFAPDAEGEYKSSYCSHMDCFRWVKRDSYLPQGSQGLKAVTQSKLGYNPIELDPELMTPYAFEKPQHLSEYSVSDAVATYYLYMKYVHPFIFSLCTIIPLNPDETLRKGTGTLCEMLLMVQAYQHNILLPNKHTDPIERFYDGHLLESETYVGGHVESLEAGVFRSDLKNEFKIDPSAIDELLQELPEALKFSVEVENKSSVDKVTNFEEIKNQITQKLLELKENNIRNELPLIYHVDVASMYPNIMTTNRLQPDSIKAERDCASCDFNRPGKTCARKLKWAWRGEFFPSKMDEYNMIKRALQNETFPNKNKFSKKKVLTFDELSYADQVIHIKKRLTEYSRKVYHRVKVSEIVEREAIVCQRENPFYVDTVKSFRDRRYEFKGLAKTWKGNLSKIDPSDKHARDEAKKMIVLYDSLQLAHKVILNSFYGYVMRKGSRWYSMEMAGITCLTGATIIQMARALVERVGRPLELDTDGIWCILPKSFPETYFFTLENGKKLYLSYPCSMLNYRVHQKFTNHQYQELKDPLNYIYETHSENTIFFEVDGPYKAMILPSSKEEGKGIKKRYAVFNEDGSLAELKGFELKRRGELQLIKNFQSDIFKVFLEGDTLEGCYSAVASVCNRWLDVLDSHGLMLEDEDLVSLICENRSMSKTLKEYEGQKSTSITTARRLGDFLGEDMVKDKGLQCKYIISSKPFNAPVTERAIPVAIFSADIPIKRSFLRRWTLDPSLEDLDIRTIIDWGYYRERLGSAIQKIITIPAALQGVSNPVPRVEHPDWLKRKIATKSGGSKRTADGSEFEPKKKRKV